(19) United States
(12) Patent Application Publication
(10) Pub. No.: US 2026/0074041 A1
(43) Pub. Date: Mar. 12, 2026

(54) METHODS OF TREATMENT AND INTERVENTIONS FOR IMPROVING OR MAINTAINING HEALTH AND WELLNESS BASED ON QUANTITATIVE ASSESSMENT OF BIOMARKER LEVELS

(71) Applicant: Matter Neuroscience Inc., Boulder, CO (US)

(72) Inventor: Axel BOUCHON, Kleinmachnow (DE)

(73) Assignee: Matter Neuroscience Inc., Boulder, CO (US)

(21) Appl. No.: 19/326,027

(22) Filed: Sep. 11, 2025

Related U.S. Application Data
(60) Provisional application No. 63/694,061, filed on Sep. 12, 2024.

(51) Int. Cl. G16H 20/00; G16H 10/60; G16H 15/00; G16H 50/70

(52) U.S. Cl. CPC G16H 20/00; G16H 10/60; G16H 15/00; G16H 50/70

(57) ABSTRACT

Provided are methods, systems including one or more processors, and computer-readable media storing instructions which, when executed by the system, cause the system to execute the method, the method including: obtaining biomarker data representing biomarker levels for different molecules for a patient; determining, based on the obtained data and using a knowledge graph linking intervention modules, target biomarker ranges, and outcomes, that at least one of the biomarker levels for the patient do not fall within the one or more target biomarker ranges; selecting, based on the obtained data and using the knowledge graph, one of the respective intervention modules associated with the biomarker level that does not fall within the target biomarker range; selecting an intervention associated with the selected intervention module; and generating, by an outputter, an output indicating the selected intervention.

### CROSS-REFERENCE TO RELATED APPLICATIONS

This application claims priority to and the benefit of U.S. Provisional Application No. 63/694,061, filed Sep. 12, 2024, the contents of which are incorporated herein in its entirety.

### FIELD

This application relates generally to treatment methods and interventions for improving health and wellness, and more specifically, to treatment methods and interventions for improving health and wellness based on quantitative assessments of biomarker levels.

### BACKGROUND

Modern healthcare practice continues to rely predominantly on pharmacological treatments as the cornerstone of managing chronic and acute conditions. While effective for symptom management and disease progression control, this reliance often sidelines other proven interventions (e.g., behavioral modification, nutritional strategies, or device-based therapies) that may work synergistically to improve long-term outcomes. Existing integrative or precision medicine initiatives have sought to tailor treatments by leveraging genetic or biochemical markers (i.e., biomarkers), but these efforts remain limited in scope-they typically emphasize a narrow slice of biomarker data and rarely integrate insights across multiple dimensions simultaneously. Biomarkers include molecular biomarkers (e.g., DNA-based, epigenetics-based, protein- and metabolite-based biomarkers), biophysical biomarkers (e.g., magnetic resonance imaging (MRI)-based, biosensor-based, physical performance-based (e.g., VO₂ max, strength)), behavioral biomarkers, and emotional biomarkers. Even advanced personalized medicine platforms often stop short of prescribing a truly dynamic, multi-modal intervention strategy that adapts over time to a patient's evolving biomarker profile. Meanwhile, clinical practice guidelines are generally siloed by specialty, with neurologists, endocrinologists, nutritionists, and behavioral health professionals working in parallel rather than in an orchestrated fashion. This fragmented landscape produces an incomplete snapshot of a patient's health, and constrains the patient's ability to achieve sustainable, long-term health and wellbeing. Consequently, there is a need for a holistic, quantitative framework that unifies diverse biomarker data and systematically escalates interventions across several domains, and methods and systems that effectively apply this framework to identify health deficiencies based on a single or few molecules or molecular networks, and coach patients to improved health and quality of life.

### SUMMARY

Disclosed herein are quantitative techniques for providing holistic, biomarker-backed, clinically proven interventions and/or treatments to patients and other healthy individuals (hereinafter, patients), which may allow patients to achieve and maintain neurological and physical health and wellness states to reach a good life. The techniques described herein may include providing a dynamic intervention plan that systematically escalates six different types of interventions—behavior, nutrition & nutritional supplement, pharmacological, medical device, surgical, genetic—with the objective to reach distinct biomarker ranges that correlate with a longer, healthier, and/or happier life. The techniques disclosed herein include applying multi-modal biomarker panels that are the first of their kind, which consider and integrate data from biochemical, biophysical, diagnostic imaging (e.g., fMRI), emotional, and behavioral biomarkers. Deep and holistic health & wellbeing assessments are acquired to correlate clinical improvements on health and wellbeing.

In some aspects, a method to improve and maintain molecular biomarker levels in a patient is provided, including: obtaining biomarker data representing a plurality of biomarker levels for a plurality of different molecules for a patient; determining, based on the obtained data representing the plurality of biomarker levels and using a knowledge graph linking one or more intervention modules, one or more target biomarker ranges, and one or more outcomes, that at least one of the plurality of biomarker levels for the patient do not fall within the one or more target biomarker ranges; selecting, based on the obtained data and using the knowledge graph, a first one of the plurality of respective intervention modules associated with the at least one biomarker level that does not fall within the target biomarker range; selecting a first intervention associated with the selected first intervention module; and generating, by an outputter, an output indicating the selected first intervention.

In some aspects, a system including one or more processors and memory storing instructions configured to be executed by the one or more processors is provided, the instructions causing the system to: obtain data representing a plurality of biomarker levels for a plurality of different molecules for a patient; determine, based on the obtained data representing the plurality of biomarker levels and using a knowledge graph linking one or more intervention modules, one or more target biomarker ranges, and one or more outcomes, whether at least one of the plurality of biomarker levels for the patient fall within the one or more target biomarker ranges; based on a determination that the at least one of the plurality of biomarker levels do not fall within the one or more target biomarker ranges, select, based on the obtained data and using the knowledge graph, a first one of the plurality of respective intervention modules associated with the at least one biomarker level that does not fall within the target biomarker range; select a first intervention associated with the selected first intervention module; and generate, by an outputter, an output indicating the selected first intervention.

In some aspects, a non-transitory computer-readable storage medium storing instructions is provided, the instructions, when executed by a system including one or more processors, causing the system to: obtain data representing a plurality of biomarker levels for a plurality of different molecules for a patient; determine, based on the obtained data representing the plurality of biomarker levels and using a knowledge graph linking one or more intervention modules, one or more target biomarker ranges, and one or more outcomes, whether at least one of the biomarker levels for the patient do not fall within the one or more target biomarker ranges; based on a determination that the at least one of the plurality of biomarker levels do not fall within the one or more target biomarker ranges, select, based on the obtained data and using the knowledge graph, a first one of the plurality of respective intervention modules associated with the at least one biomarker level that does not fall within the target biomarker range; select a first intervention associated with the selected first intervention module; and generate, by an outputter, an output indicating the selected first intervention.

### BRIEF DESCRIPTION OF THE FIGURES

The invention will now be described, by way of example only, with reference to the accompanying drawings, in which:

FIG. 1A shows a longevity function quantified based on healthy life years and total life span, in accordance with some embodiments.

FIG. 1B shows a quantification of Good Life based on health, happiness, and longevity (e.g., happy life years, healthy life years, and life span), in accordance with some embodiments.

FIG. 2A shows an exemplary knowledge graph linking interventions/treatments, target biomarker ranges, and clinical and/or wellbeing outcomes, in accordance with some embodiments.

FIG. 2B shows another exemplary knowledge graph linking interventions/treatments, target biomarker ranges, and clinical and/or wellbeing outcomes, in accordance with some embodiments, the knowledge graph in FIG. 2B including additional nodes and connections as compared to the knowledge graph in FIG. 2A.

FIG. 3A shows a process diagram for generating a health report that identifies treatments and/or interventions for maximizing good life using biomarker data of a patient or healthy individual, in accordance with some embodiments.

FIG. 3B shows a plurality of modules that may be used to create the report using the process shown in FIG. 3A, in accordance with some embodiments.

FIG. 4 shows a flowchart representation of a method for assessing biomarkers and determining and applying treatments and/or interventions based on the biomarker assessment, in accordance with some embodiments.

FIG. 5 shows example addiction and toxicology entry criteria in accordance with some embodiments.

FIG. 6 shows exemplary dependency assessments and toxicology for illegal and legal recreational drug usage, in accordance with some embodiments.

FIG. 7 shows exemplary dependency assessments and toxicology for off-label and on-label medical drug usage, in accordance with some embodiments.

FIG. 8 shows exemplary dependency assessments and toxicology for fast food and sweets/sugar consumption, in accordance with some embodiments.

FIG. 9 shows exemplary dependency assessments and toxicology for behavioral addictions, in accordance with some embodiments.

FIG. 10 shows output data generated by the various addiction and toxicology entry criteria from FIGS. 5-9 as inputs to an inclusion/exclusion decision matrix, in accordance with some embodiments.

FIG. 11 shows an exemplary inclusion/exclusion decision matrix, in accordance with some embodiments.

FIG. 12 shows various subcategories of a health and wellness assessment, including categories for assessing health and disease as well as good life wellness markers, in accordance with some embodiments.

FIG. 13 shows an exemplary stratification matrix that may be used for selecting one or more intervention modules (and/or interventions therein), and that may be used to select a sequence of intervention modules (and/or interventions therein) based on the health and wellness assessment data, in accordance with some embodiments.

FIG. 14 shows a matrix guiding tool selection based on a selection of different modules, in accordance with some embodiments.

FIG. 15 shows types of data that may be monitored and gathered by the various tools that may be selected in FIG. 14, in accordance with some embodiments.

FIG. 16 shows a plurality of intervention modules that may be selected for maximizing the good life function, in accordance with some embodiments.

FIG. 17 shows physiological need modules from FIG. 16, including hydration, metabolic health, and physiological composition modules, in accordance with some embodiments.

FIG. 18 shows additional physiological need modules from FIG. 16, including sleep and mental composition modules, in accordance with some embodiments.

FIG. 19 shows hedonic need modules from FIG. 16, including basic motivation, sexual wellbeing, and interpersonal self-esteem modules, in accordance with some embodiments.

FIG. 20 shows additional hedonic need modules from FIG. 16, including self-confirmed self-esteem and pleasure modules, in accordance with some embodiments.

FIG. 21 shows eudaimonic need modules from FIG. 16, including family/contentment, friends/amusement, and gratitude/mindfulness modules, in accordance with some embodiments.

FIG. 22 shows additional eudaimonic need modules from FIG. 16, including intrinsic motivation and extrinsic motivation modules, in accordance with some embodiments.

FIG. 23 shows processes for training, deploying, and refining a personalized and universal AI models, according to some embodiments.

FIG. 24 shows an exemplary computer system that can be used to execute the methods for determining interventions/treatments that maximize health and wellbeing as described herein, in accordance with some embodiments.

FIG. 25 shows a prophetic example of identifying a deep sleep deficiency based on biomarker data and selecting and carrying out a sleep intervention to improve the deep sleep biomarker, in accordance with some embodiments.

FIG. 26 shows escalated interventions of the sleep module for improving the deep sleep biomarker as introduced in FIG. 25, in accordance with some embodiments.

FIG. 27 shows additional escalated interventions of the sleep module for improving the deep sleep biomarker as discussed in FIGS. 25 and 26, in accordance with some embodiments.

### DETAILED DESCRIPTION

Disclosed herein are systems, methods, and computer-readable storage media for delivering holistic, biomarker-driven interventions designed to help patients or healthy individuals (collectively referred to hereinafter as patients) achieve and sustain neurological and physical health, wellness, and improve overall quality of life. The systems and methods described herein may leverage a multi-modal knowledge graph that integrates and associates various types of biomarker data, including biochemical, biophysical, diagnostic imaging (e.g., MRI), emotional and behavioral data to generate a dynamic and personalized health profile and intervention for each patient. The disclosed systems and methods may improve upon existing approaches that rely predominantly on pharmacological treatments or narrow biomarker panels by utilizing expansive biomarker panels, gathered from, e.g., surveys, GUI-based data collection, brain imaging, and wearable continuous monitoring devices, enabling a comprehensive assessment of health trajectories.

Using the biomarker data, the systems and methods described herein can generate a dynamic intervention plan that systematically escalates six different categories of interventions, including behavioral, nutritional and supplement-based, pharmacological, device-based, surgical, and genetic. This escalation pathway can be designed to guide patients toward achieving biomarker ranges that correlate with measurable improvements in longevity, neurological function, physical vitality, and subjective wellbeing. The systems and methods described herein can utilize one or more artificial intelligence (AI)-based agents, rule sets, or combinations thereof to process the biomarker data and determine the intervention(s) that can lead to improved health and wellbeing, in turn optimizing intervention sequencing and adaptation over time. The patient's progress through interventions can be easily monitored using continuous monitoring tools (as mentioned above) and validated against target ranges during the treatment. Overall, by uniting diverse biomarker data streams with a structured, adaptive knowledge graph, the systems and methods described herein may provide a comprehensive solution for addressing the current limitations of fragmented and pharmacologically biased healthcare systems.

In the following description of the various examples, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

### Function Quantifying Health and Wellbeing

In some embodiments, the techniques disclosed herein may include applying gathering data and applying said data to maximize a function that quantifies health and wellbeing, referred to hereinafter as a Good Life (GL) function. As shown conceptually below, quantification of a Good Life may be represented as a function of longevity, healthy life years, and happy life years:

GL=f(longevity,healthy life years,happy life years)

Happy life years may be identified and quantified based on a neuroscientific definition, for example as provided by the Matter score (see, e.g., U.S. Pat. No. 12,207,927 (referred to as a “return on happiness (ROH)” score), which is hereby incorporated by reference in its entirety), which is a function of neurotransmitters for six neurotransmitters associated with positive emotional intensity and good memories.

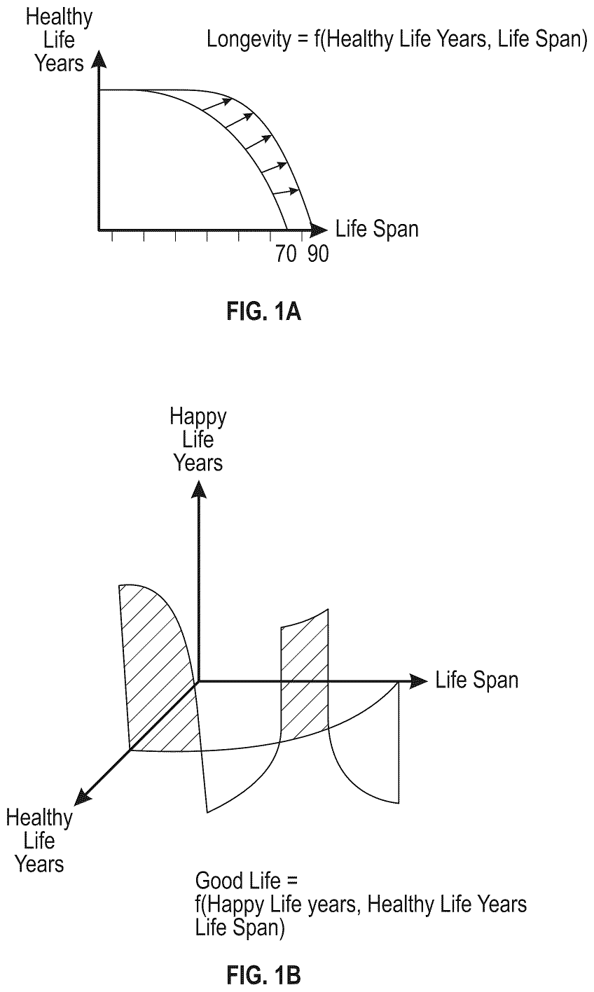

As shown in FIG. 1A, a longevity function may be quantified based on healthy life years and total life span. As shown in FIG. 1B, a quantification of a Good Life may be based on health, happiness, and longevity (e.g., happy life years, healthy life years, and life span).

### Determining Interventions to Maximize Quantification of Health and Wellbeing

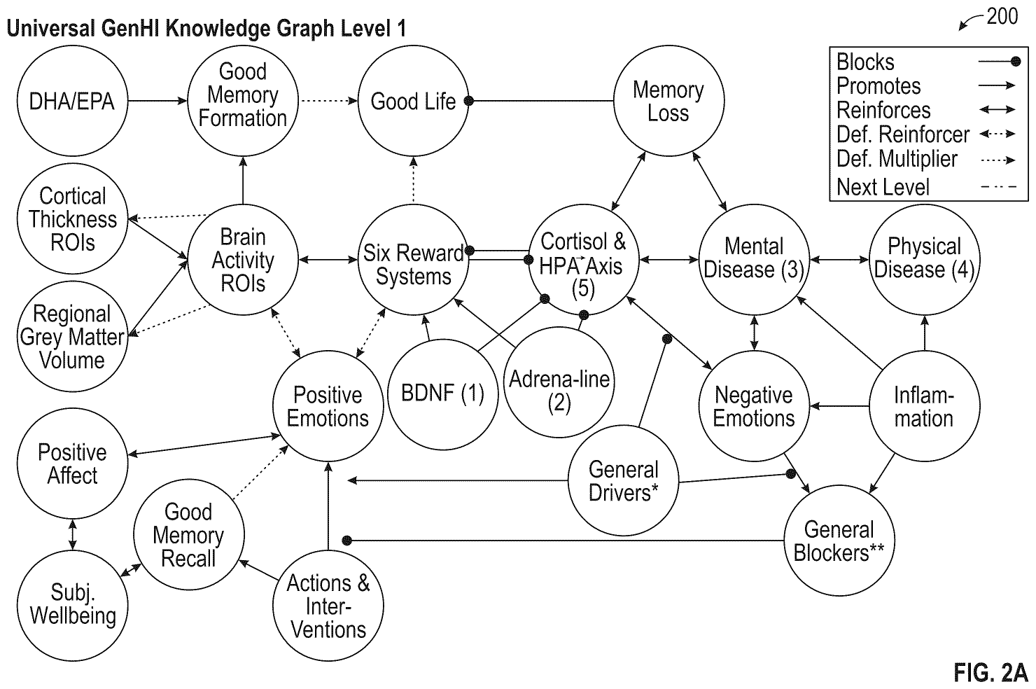

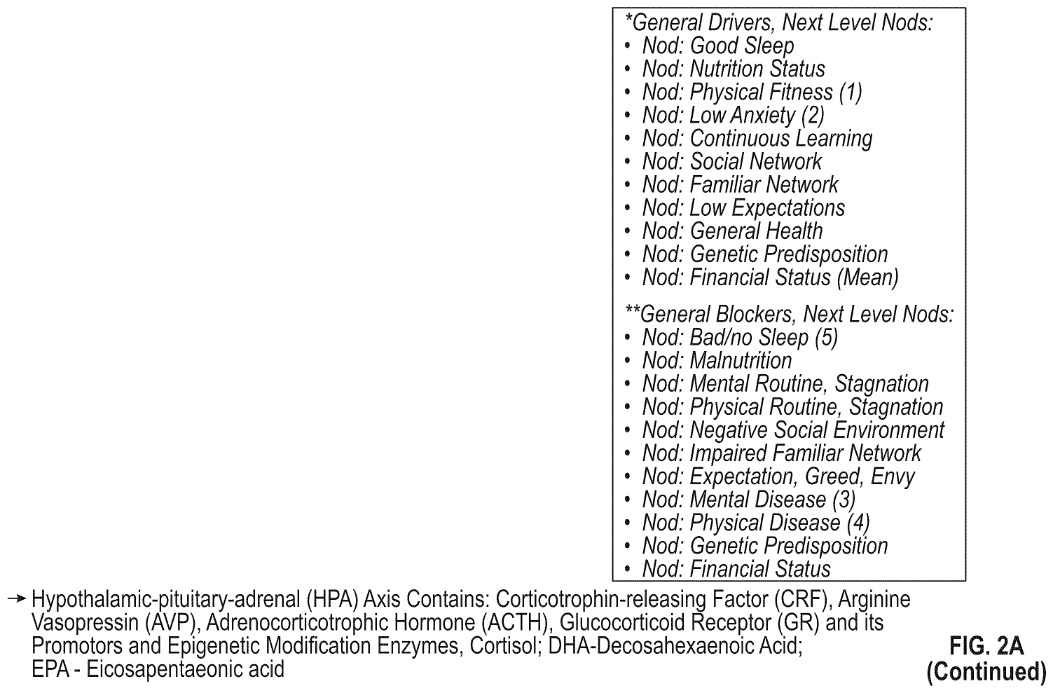

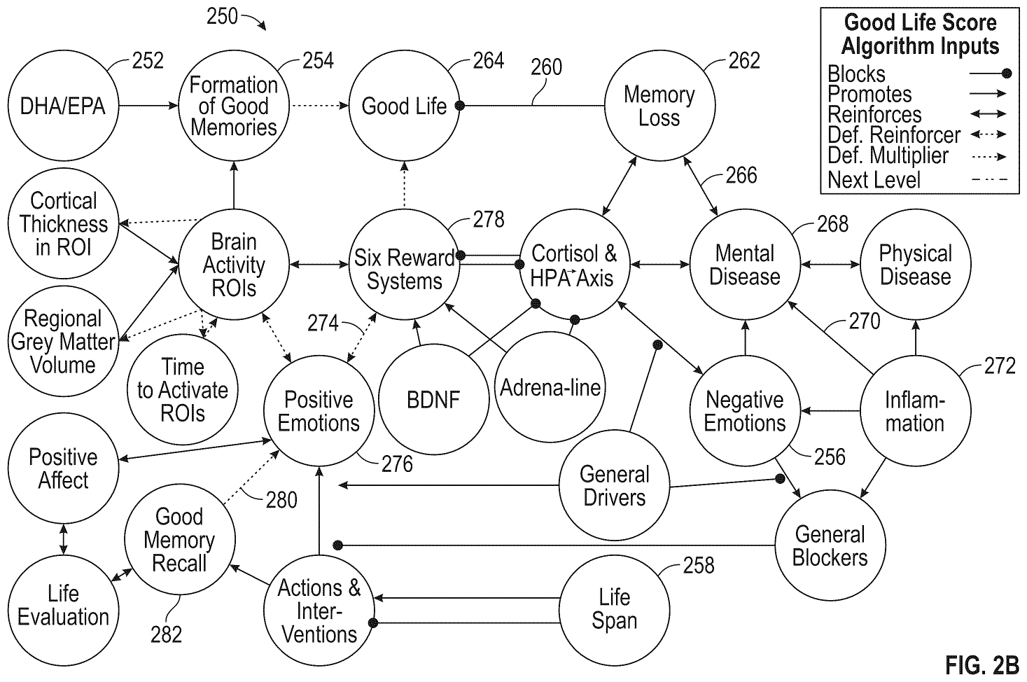

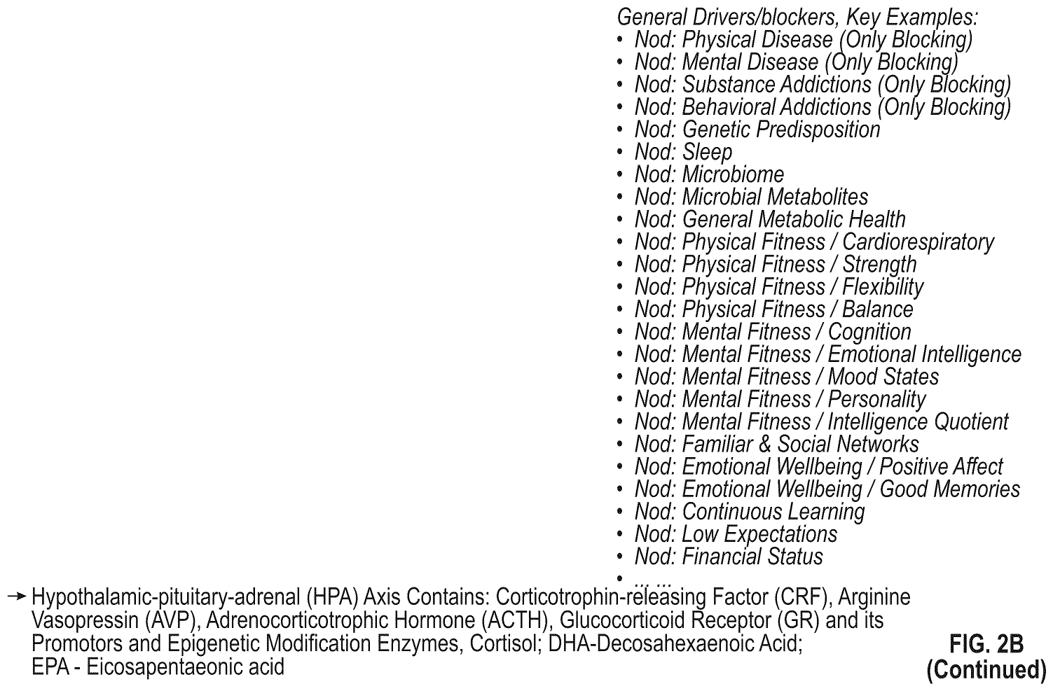

In some embodiments, a health and wellbeing function (e.g., the GL function) may take the form of a large knowledge graph, which may link interventions, target biomarker ranges, and clinical and/or wellbeing outcomes. FIGS. 2A-2B show examples of such knowledge graphs 200, 250, respectively, that interlink interventions/treatments, target biomarker ranges, and clinical and/or wellbeing outcomes. In some examples, the knowledge graph links only molecules and biomarkers (and their target ranges). In some examples, the knowledge graph includes intervention modules that are linked to molecules (and their respective biomarkers). In some examples, the knowledge graph includes interventions grouped within intervention modules, the interventions linked to the intervention modules. In some examples, the knowledge graph includes clinical and/or wellbeing outcomes linked to intervention modules (that are linked to molecules, and in turn their respective biomarkers). The knowledge graph can include a layered structure of node types (e.g., intervention nodes, biomarker nodes, molecule nodes, outcome nodes, etc.). Various node types can be linked to one another, as described in greater detail below, using different types of links demonstrating the relationship between interventions, biomarkers, molecules, and/or outcomes.

In some embodiments, a knowledge graph for use as described herein may be generated by artificial intelligence (AI) based on data sets demonstrating how interventions influence the key ranges for the relevant biomarkers. As described herein, the knowledge graph can include a plurality of nodes and links between nodes, one or more sets of biomarkers mapped to each of the nodes in the knowledge graph (as described in greater detail below). In some examples, AI may refer to an inference engine that applies logical rules to a knowledge base/graph. In some examples, a knowledge graph as described herein can be used to train one or more AI agents, or models. The techniques described herein may enable creation of an inference engine that is used as an AI expert system to guide patients to outcomes that maximize quantifications of health and wellbeing (GL function).

A treatment method and/or system for treatment may maximize the GL function (e.g., the knowledge graph) to determine and generate outputs indicating interventions that will guide a user to maximizing their health and wellbeing over the course of their life, thus allowing users to live the best life. This maximization may be quantitative and based on scientifically backed biomarkers, and it may allow for attaining a balance between a quantifiably long, quantifiably healthy, and quantifiably happy life, including by navigating scenarios in which increasing or decreasing certain aspects of health and wellness can be synergistic or contradicting.

In some embodiments, a proprietary protocol and/or clinical studies may be used to gather data from a patient regarding at least ten molecules (or sets of molecules) that can be quantifiably linked to happiness and wellbeing. In some examples, data regarding more than ten individual molecules, such as up to 13 individual molecules that can be quantifiably linked to happiness and wellbeing may be gathered. The molecules may include, but are not limited to: dopamine, testosterone, estrogen (which in some embodiments may be collectively referred to and/or quantified with testosterone as a testosterone level), serotonin, oxytocin, arachidonoyl ethanolamine (AEA), 2-archidonoyl glycerol (2-AG) (which in some embodiments may be collectively referred to and/or quantified with AEA as cannabinoids), beta-endorphin (which in some embodiments may be referred to as opioids), cortisol, adrenaline (e.g., noradrenalin), brain-derived neurotrophic factor (BDNF), eicosatetraenoic acid (EPA), and docosahexaenoic acid (DHA).

In some examples, the first six of the above molecules (or sets of molecules) (i.e., dopamine, testosterone, serotonin, oxytocin, cannabinoids, and opioids) may be used to compute a quantification of brain plasticity and good memories, otherwise referred to herein as a Matter Score.

In some examples, biomarker data for a patient includes data regarding one or more molecules involved in mitochondrial energy metabolism (e.g., sirtuins, AMP-activated protein kinase). In some examples, biomarker data for a patient includes data regarding one or more molecules involved in neuronal activation, enhanced cognition, and memory formation (e.g., NMDA receptors, such as ligands thereof (e.g., glutamate, glycine)). In some examples, biomarker data for a patient includes data regarding one or more molecules involved in regulating acute and chronic inflammations (e.g., nutrients, such as magnesium, vitamin D, vitamin E, zinc, selenium; metabolites reducing stress, such as NAD+; polyphenol targets, such as phospholipase A2 or lipoxygenase). In some examples, biomarker data for a patient includes data regarding one or more molecules involved in tumor suppression or activation (e.g., p53, BRAC1). The above-listed molecules are merely exemplary and are not intended to limit the scope of the molecules that may be quantifiably linked to happiness and wellbeing, as described herein.

In some examples, biomarker data for a patient includes data regarding one or more molecules identified using one or more of metabolomic biomarker panels, fMRI and/or PET-tracer enhanced imaging techniques, epigenetics, blood biomarker panels, transcriptomics, proteomics, microbiomics, and genomics (collectively, “OMICs”), and/or early cancer biomarker panels. In some examples, one or more molecules involved in mitochondrial energy metabolism may be identified and/or confirmed using at least one of OMICs, metabolomic biomarker panels, and PET-tracer enhanced imaging techniques. In some examples, one or more molecules involved in neuronal activation, enhanced cognition, and memory formation may be identified and/or confirmed using at least one of epigenetics, fMRI, and PET-tracer enhanced imaging techniques. In some examples, one or more molecules involved in regulating acute and chronic inflammations may be identified and/or confirmed using at least one of blood biomarker panels, OMICs, and genomics tests. In some examples, one or more molecules involved in tumor suppression or activation may be identified and/or confirmed using at least one of early cancer biomarker panels identifying key loss-of-function mutations and genomics tests.

As described herein, a health and wellbeing assessment may be applied along an entire process as explained herein, and may span clinical, health, and wellness aspects. The health and wellbeing assessment may rely on data gathered from research, clinical questionnaires, 7T MRI brain scans, epigenetics, genetics, and/or a specific set of blood biomarkers that monitor for one or more of the molecules listed above. In some examples, the assessment includes at least one of clinical intake records, biomarker panels, advanced imaging results, physical fitness assessments, wearable device metrics, psychological surveys, and/or behavioral surveys.

In some embodiments, continuous monitoring and/or gathering of data may be performed using one or more tools, such as a survey and/or automated GUI-based data collection (e.g., via Matter App, as described in U.S. Pat. No. 12,207,927) to which a user can enter memory and/or emotion information that may be ingested by the systems described herein to determined (e.g., calculate) neurotransmitter levels and/or neurotransmitter needs. In some embodiments, data monitoring and/or gathering can additionally or alternatively be performed using a patch or other wearable device configured for monitoring blood glucose levels. In some embodiments, data monitoring and/or gathering can additionally or alternatively be performed using a patch or other wearable device configured for monitoring cortisol levels. In some embodiments, data monitoring and/or gathering can additionally or alternatively be performed using a fitness tracker or other wearable (e.g., an electronic ring, watch, band, or other similar device) configured for monitoring sleep quality, physical activity levels, stress levels, and/or emotional states correlating with high arousal states. In some embodiments, data monitoring and/or gathering can additionally or alternatively be performed using a wearable device (e.g., an electronic ring, watch, band, or other similar device) configured for monitoring movement data, elevation data, and/or co-location or proximity data with other persons.

In some embodiments, biomarker data can include at least one of mental disease biomarker data, mental fitness biomarker data, emotional wellbeing biomarker data, sleep biomarker data, metabolic fitness biomarker data, and/or physical fitness biomarker data. The data can include quantitative data, qualitative data (e.g., a ranking), and/or categorical data (e.g., binary yes/no). The biomarker data may be gathered using a self-assessment. In some examples, the biomarker data are additionally or alternatively gathered using one or more molecular tests. In some examples, the biomarker data are additionally or alternatively gathered using one or more biophysical tests.

As an example, mental disease biomarker data may include, but are not limited to, data regarding a patient's state of depression, state of anxiety, state of cognition (e.g., presence of Alzheimer's disease), substance abuse, toxicology from substance abuse, and/or behavior addictions. As another example, mental fitness biomarker data may include, but are not limited to, data regarding a patient's mood, personality, emotional intelligence, intelligence quotient (IQ), and/or Raven Intelligence Test (e.g., determined using 7T fMRI, as described herein). As another example, emotional wellbeing biomarker data may include, but are not limited to, data regarding a patient's subjective wellbeing, Matter score, semantic neurofeedback (e.g., determined using 7T fMRI), and/or cortical thickness (e.g., determined using 7T fMRI). As another example, sleep biomarker data may include, but are not limited to, data from a patient's Pittsburgh Sleep Quality Index (PSQI) self-assessment and/or biophysical sensor data (e.g., gathered from a smart watch, band, ring, or other wearable device). As another example, metabolic biomarker data may include, but are not limited to, data related to a patient's blood, urine, and/or microbial metabolites. As another example, physical fitness biomarker data may include, but are not limited to, data regarding a patient's body composition (e.g., determined using dual-energy X-ray absorptiometry, or DEXA), strength, flexibility, balance, cardiorespiratory, and/or biophysical sensor data (e.g., gathered from a wearable device as described herein).

In some examples, the biomarker data includes one or more overarching biomarker panels. For example, the biomarker data may include data regarding a patient's clinical anamnesis, genomics, epigenetics, microbiomics, OMICs (e.g., genomics, transcriptomics, and/or proteomics), magnetic resonance imaging (MRI) results (e.g., 3T clinical brain imaging results, 7T functional and structural MRI results), and/or cancer testing results (e.g., cell-free DNA cancer testing results).

At least a portion of the plurality of biomarkers (e.g., types of biomarkers, or specific biomarkers) may be mapped to one or more of the nodes of the knowledge graph (e.g., knowledge graph 200, 250). For example, one or more categories of biomarkers, or specific biomarkers, may be mapped to a given node, and another, different category of biomarkers (or different specific biomarkers) may be mapped to another node. In some examples, the same category of biomarkers (or specific biomarkers) may be mapped to more than one node in the knowledge graph. In this way, when biomarker data is provided to the system, it can methodically process the biomarker data using the knowledge graph. For example, with reference to knowledge graph 250 shown in FIG. 2B, biomarker data regarding a patient's metabolic fitness (e.g., blood data) may be mapped to a DHA/EPA node 252 of the knowledge graph 250. In some examples, biomarker data regarding a patient's emotional wellbeing (e.g., Matter score, semantic neurofeedback data) may be mapped to a formation of good memories node 254 of the knowledge graph 250. In some examples, biomarker data regarding a patient's mental disease (e.g., depression) and/or emotional wellbeing (e.g., subjective wellbeing, semantic neurofeedback data) may be mapped to a negative emotions node 256 of the knowledge graph 250. In some examples, biomarker data regarding a patient's epigenetics, metabolic fitness (e.g., blood data), and/or physical fitness (e.g., body composition) may be mapped to a lifespan node 258 of the knowledge graph 250.

In some examples, one or more types of biomarker data (or specific biomarkers) may be considered at substantially all of the nodes of the knowledge graph. For example, biomarker data regarding a patient's mental disease (e.g., substance abuse, toxicology data) may be considered at substantially all nodes. In some examples, the overarching biomarker panels described herein (e.g., genomics, epigenetics, and microbiomics data, to name a few) may be considered at all nodes.

One or more AI agents may traverse the knowledge graph to optimize the patient's good life function. The knowledge graph may include one or more link types for linking various nodes for traversing the knowledge graph. The different link types may be traversed differently from one another in using the knowledge graph to determine whether biomarker levels meet target levels and/or select interventions using the knowledge graph. In some examples, a knowledge graph may include one or more blocking links, promoting links, reinforcing links, definite reinforcing links, and/or definite multiplying links between sets of nodes. For example, with reference to knowledge graph 250 in FIG. 2B, the knowledge graph 250 includes a blocking link 260 extending from a memory loss node 262 to a good life node 264, indicating that memory loss blocks good life. The knowledge graph 250 may include a reinforcing link 266 between the memory loss node 262 and a mental disease node 268, indicating that memory loss and mental disease reinforce one another. The knowledge graph 250 may include a promoting link 270 extending from an inflammation node 272 to the mental disease node 268, indicating that inflammation promotes mental disease. The knowledge graph 250 may include a definite reinforcing link 274 (indicated using dashed lines) extending between a positive emotions node 276 and a reward systems node 278, indicating that positive emotions strongly reinforce the reward systems, and vice versa. The knowledge graph 250 may include a definite promoting link 280 (indicated using dashed lines) extending from a good memory recall node 282 to the positive emotions node 276, indicating that good memory recall promotes positive emotions.

In some examples, the links between nodes may be weighted. For example, a definite reinforcing link (e.g., link 274) between nodes may be weighted higher than a simple reinforcing link (e.g., link 266). Likewise, a definite promoting link (e.g., link 280) may be weighted higher than a simple promoting link (e.g., link 270). In some examples, the links may be weighted with a scale of strengths. For example, reinforcing links may be either a strong reinforcing link, a medium reinforcing link, or a weak reinforcement link. A score may be associated with each link weight (e.g., on a scale between 0 and 1). In some examples, the weights assigned to the links of the graph may be updated over time based on a feedback loop that applies data corresponding to one or more updated biomarker levels based on treatment and/or intervention recommendations generated by the AI agent and resultant outcomes (e.g., executed interventions). The feedback loop can be used to adjust the knowledge graph by, for example, adding or removing a link, adjusting a link type, and/or adjusting a weighting of a link. In this way, the knowledge graph may determine different interventions from one patient to the next based on the weights of the links in the knowledge graph.

As described in greater detail below, nodes of the knowledge graph (and/or variables associated with the nodes) may be initially weighted for the AI agent to assess the importance of the biomarkers in determining good life function. In some examples, the weights assigned to the nodes/variables of the graph may be updated over time based on a feedback loop that applies data corresponding to one or more updated biomarker levels based on treatment and/or intervention recommendations generated by the AI agent and resultant outcomes (e.g., executed interventions). The feedback loop can be used to adjust the knowledge graph by, for example, adding or removing a node and/or adjusting a weighting of a node. In this way, the knowledge graph may determine different interventions from one patient to the next based on the weights of the nodes or associated variables in the knowledge graph.

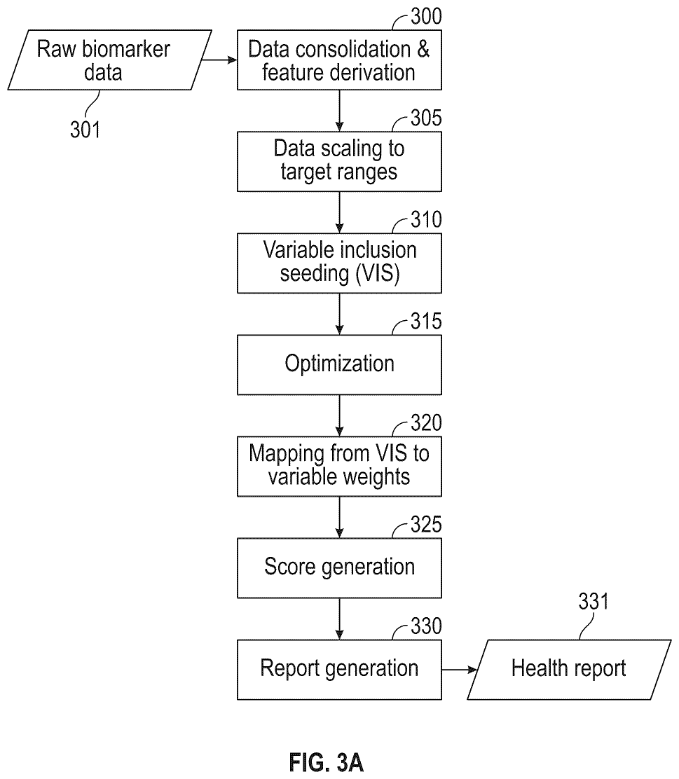

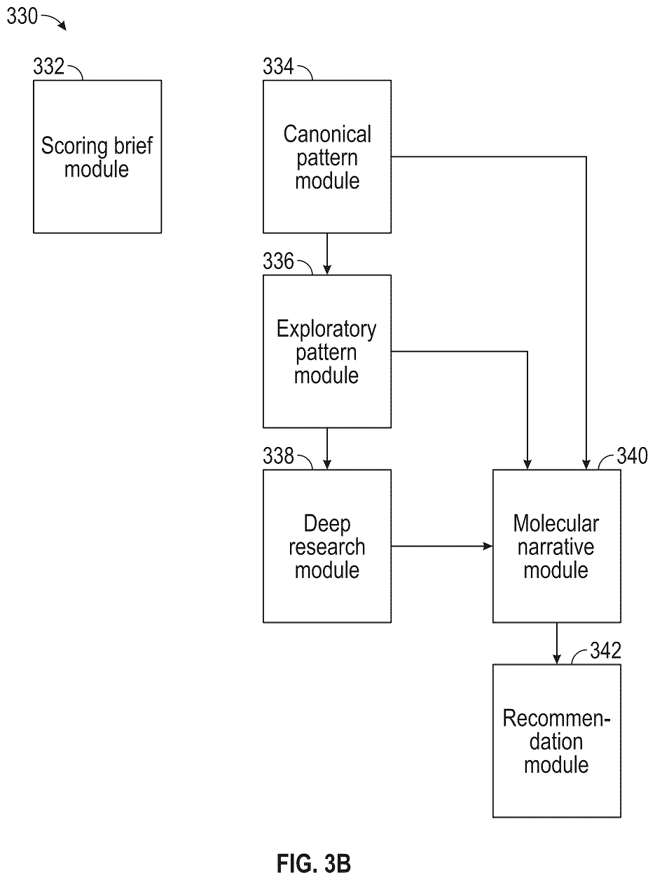

FIGS. 3A-3B show process diagrams for generating a health report that identifies treatments and/or interventions for maximizing good life using biomarker data of a patient. In particular, FIG. 3A shows an overall process diagram for processing biomarker data and biomarker data fields in relation to the ten molecules for holistically assessing a patient's health and wellbeing and determining treatments and/or interventions to maximize the patient's good life. FIG. 3B shows modules of the report generation portion of the process in FIG. 3A.

At block 300, biomarker data 301 may be processed by a computing system and stored as numerical scores for further processing. Variables (i.e., biomarker types) suitable for numeric analysis may be identified and tagged. For example, structured, scoring-compatible data may be separated from descriptive or narrative entry data.

At block 305, the biomarker data may be scaled to biological target ranges. For example, using an AI model (e.g., an LLM model or similar), clinical reference anchors and ranges may be extracted. In some examples, anchors and ranges can be extracted using a prioritized sequence fed to the AI model. The sequence may prioritize embedded scoring notes from the dataset, followed by clinical norms, followed by participant data plausibility checks, and finally, followed by trusted external references. The biomarker data may be monotonically transformed (e.g., linear or logarithmically), based on each variable's distribution. In some examples, rank-based logic is applied to transform the data. In some examples, scaling the biomarker data includes clamping out-of-range values to maintain comparability. In some examples, one or more reference anchors may be individually adjusted, for example, in the instance participant-specific factors (e.g., age, sex) require distinct clinical norms.

At block 310, an AI-based rubric may be generated for systematically assigning initial variable inclusion scores (VIS). The VIS can reflect each variable's anticipated relevance to a specific molecule. In some examples, the AI agent may apply the rubric for each of the above-identified Good Life molecule to assign each variable a relevance tier based on the variable's relationship with the molecule. The tiers may range between a direct mechanistic link and an unrelated or unknown connection. In some examples, one of six tiers may be assigned to each variable. The tiers may include, but are not limited to, (1) direct mechanistic link (strong evidence), (2) direct mechanistic link (moderate/adjacent evidence), (3) behavioral/physiological correlate, (4) general proxy measure, (5) weak or unclear association, and (6) unrelated or unknown connection. Each of the tiers may be associated with a fixed numeric weight that can be assigned to the variable as the VIS. For example, in accordance with the above-listed tiers, the weights may range between 1 and 0, such as between 0.70 and 0.00. In some examples, the weights assigned to the respective tiers are 0.70, 0.55, 0.40, 0.30, 0.10, and 0.00. In some examples, in addition to the weights, the rubric can include directional labels that reflect each variable's relationship to each Good Life molecule. The directional labels may include, but are not limited to, positive, negative, and unknown. In some examples, an AI model (e.g., an LLM) is used to assign directional labels.

At block 315, the VIS may be optimized, for example, using a machine learning-based approach. Optimizing the VIS scores may include defining a plurality of hyperparameters for managing VIS updates. Hyperparameters may include, but are not limited to, learning rate, scaling factor, and dampening exponent. Hyperparameters can increase the likelihood of stable and incremental adjustments. In some examples, optimizing the VIS may include assessing biomarker data of one or more participants by temporarily excluding the patient's data, and predicting the patient's molecular state from remaining patient data. In some examples, for variables meeting statistical threshold (e.g., minimum z-scores for variable and tone strength), the VIS can be updated to reduce the influence of noise.

At block 320, the VIS may be algorithmically mapped to variable weights. In some examples, mapping VIS to variable weights may include assigning non-negative weight magnitudes to variables whose VIS exceeded the statistical threshold (described above). In some examples, the previously assigned directional labels can be used to convert each magnitude into a signed contribution. In some examples, mapping VIS to variable weights can include performing redundancy pruning (e.g., via Spearman correlation, such as >0.85). In this way, highly correlated variables can be identified, and the influence therefrom can be reduced by retaining only the variable(s) with the highest VIS. In some examples, mapping the VIS to variable weights can include applying configurable scaling (e.g., weight-power exponent) to the VIS and enforcing a maximum cap to balance contributions and prevent excessive dominance by individual variables. In some examples, mapping the VIS to variable weights can include normalizing the final weights. Normalizing the weights can ensure consistent interpretation and meaningful comparison across molecules and/or participants.

The weights assigned to the variables (and, thus, to the nodes of an associated knowledge graph) may be updated periodically during use of the system. In some examples, weights are updated in real-time, as the AI agent is continuously trained using ingested patient data.

At block 325, a patient's composite good life score may be determined using the final weights from above. In some examples, the composite score is determined by finding the dot product of the scaled biomarker data and the final normalized weights of the variables. In some examples, determining a composite score can include applying a transformation (e.g., a logistic-linear blended transformation) to the raw composite score, in turn converting the raw score into understandable percentile values. In some examples, non-linear logistic mapping is applied in a mid-score range to accentuate any subtle differences between patients scoring near average. In some examples, linear mapping is maintained at distribution extremes (e.g., the tail ends of the data) to preserve accurate differentiation of extremely high and low molecular states, without distortion.

At block 330, the AI system may generate a health report that details the patient's molecular status, health meaning, and/or next-step guidance. For example, the health report may indicate one or more determined interventions and/or treatments. FIG. 3B indicates a plurality of modules (or nodes) that may be used to create the report.

At module 332, generating the health report may include using at least one AI agent to generate a scoring brief. The scoring brief may summarize the top-weighted variables for each molecule. In some examples, the scoring brief module 332 can provide concise, molecule-specific explanations.

At module 334, generating the health report may include using at least one AI agent to detect canonical patterns, i.e., well-known physiological patterns in the patient's data set. The canonical pattern module 334 may explain how the detected patterns reflect Good Life molecule function and/or imbalance.

At module 336, generating the health report may include using at least one AI agent to flag findings not covered by the canonical pattern module 334. The exploratory pattern module 336 may propose one or more biologically plausible hypotheses to explain the findings not covered, and evaluate whether one or more of the findings warranted a deeper study. Based on a determination that additional research is needed, the module 334 may generate a focused literature search prompt. When research is not needed, the module 334 may generate a concise conclusion integrating existing signals.

At module 338, generating the health report may include using at least one AI agent to, for the portion of exploratory patterns requiring additional research, run evidence-first literature searches. The deep research module 338 may produce structured, mechanism-focused deep dives that can be fed back into the narrative of the health report.

At module 340, generating the health report may include using at least one AI agent to tie together the canonical patterns, exploratory patterns, and deep-research insights into a single report for a given patient. The molecular narrative module 340 may include context-aware guidance in the narrative for improving molecules and/or overall health status.

At block 342, generating the health report may include using at least one AI agent to generate high-leverage, personalized next step recommendations. The recommendations can be explicitly tied to the narrative's key findings and/or molecular priorities. The recommendations can indicate one or more treatments and/or interventions for the patient.

In some embodiments, a method of diagnosis, treatment, and/or intervention may include application of one or more of a plurality (e.g., 17) of physiological modules that have been clinically proven to influence health and wellbeing, and/or that have been scientifically proven to influence key molecules (e.g., those listed above) that may be quantifiably linked to human wellbeing. In some examples, one or more (e.g., each) module may pertain to a specific panel of one or more associated biomarkers and one or more associated treatments/interventions.

In some embodiments, the application of treatments and/or interventions as disclosed herein may be structured such that those treatments and/or interventions are applied in an order of escalating invasiveness. Less invasive treatments/interventions may be applied before an assessment is made as to whether a more invasive treatment/intervention should be applied. Treatments/interventions may be stepwise enhanced if optimal target ranges for associated biomarkers for a certain panel are not met. In some embodiments, an escalating treatment/intervention sequence may be: (1) behavioral; (2) nutritional supplements; (3) pharmacological; (4) medical device; (5) surgical; and (6) genetic.

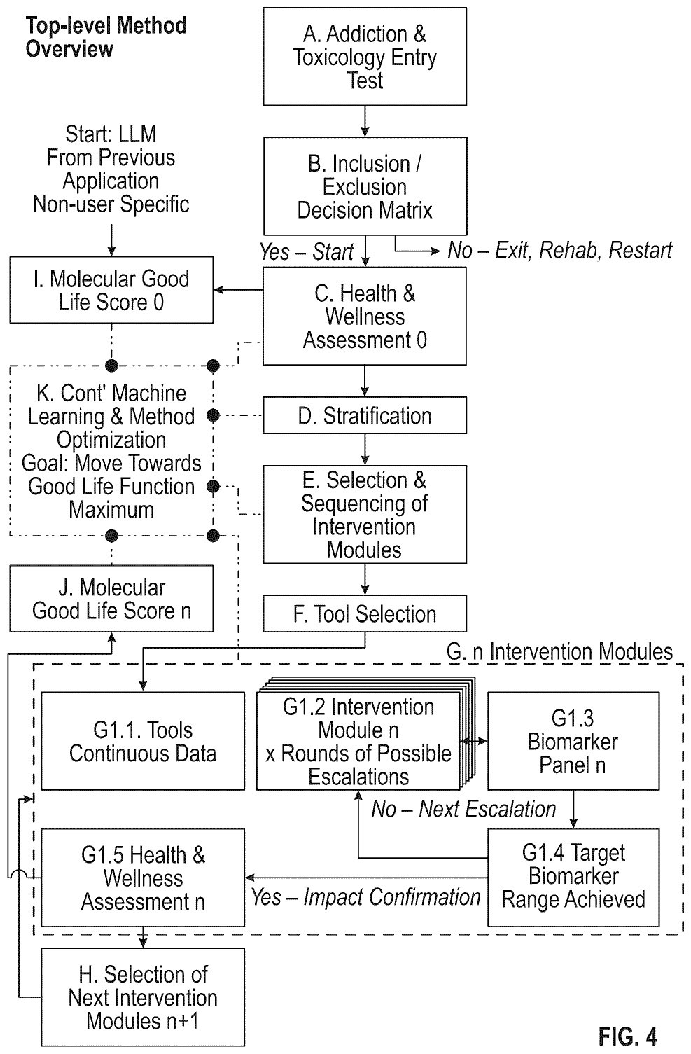

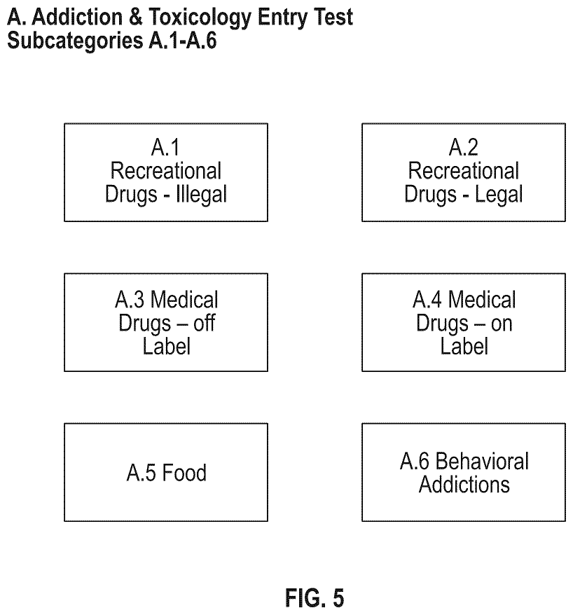

FIG. 4 shows a flowchart representation of a method for assessing biomarkers and determining and applying treatments and/or interventions based on the biomarker assessment, in accordance with some embodiments. As shown, a patient may first be subjected to addiction and toxicology screening tests and/or other exclusion criteria (e.g., relating to recreational drugs, medical drugs, food, and behavioral addictions) at blocks A and B. FIG. 5 shows example addiction and toxicology entry criteria, in some embodiments. In some examples, addiction and toxicology screening tests may include tests for illegal recreational drug usage (A1), legal recreational drug use (A2), off-label medical drug usage (A3), on-label medical drug usage (A4), food consumption (A5), and/or behavior addictions (A6).

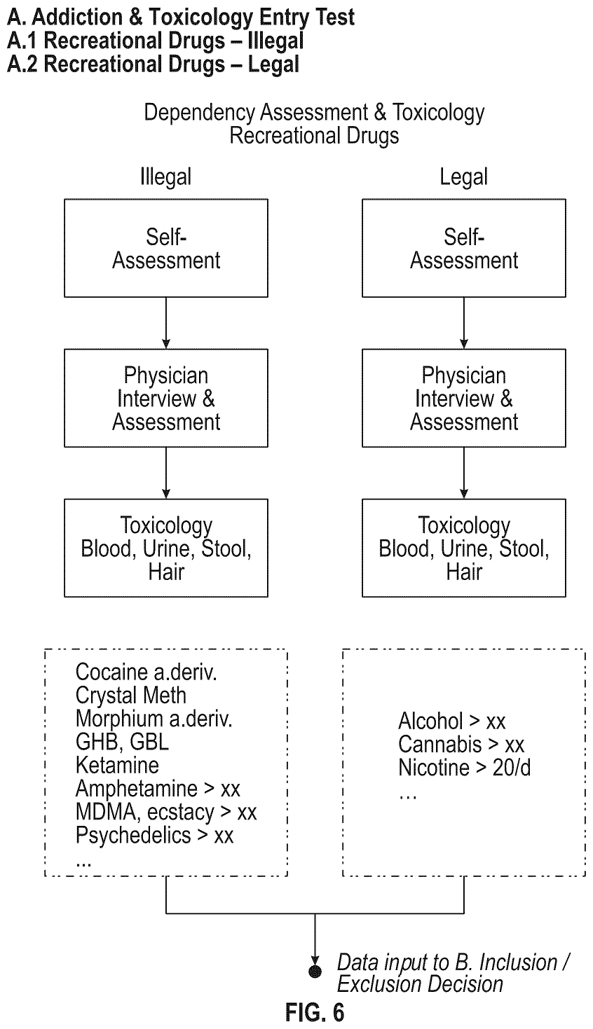

FIGS. 6-9 show details for assessments of various addiction and toxicology entry criteria, in some embodiments. The results of the assessments can be provided as input to the inclusion/exclusion decision matrix at B in FIG. 4. FIG. 6 shows exemplary dependency assessments and toxicology for illegal and legal recreational drug usage. Assessments can include self-assessment, followed by physician interview and assessment. Following assessments, toxicology may include blood, urine, stool, and/or hair toxicology. Exemplary illegal and legal recreational drugs are provided in FIG. 6.

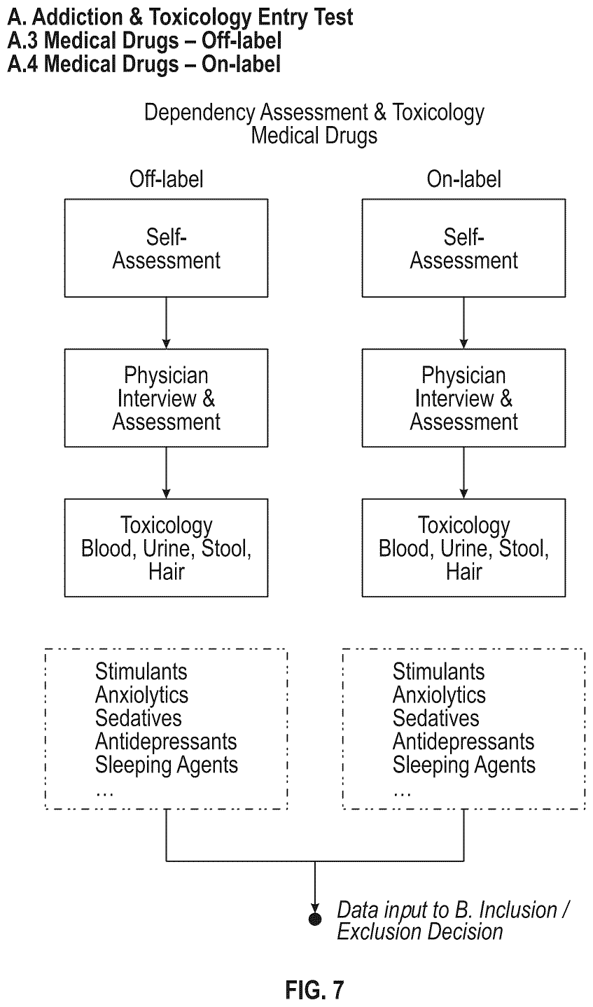

FIG. 7 shows exemplary dependency assessments and toxicology for off-label and on-label medical drug usage. Assessments can include self-assessment, followed by physician interview and assessment. Following assessments, toxicology may include blood, urine, stool, and/or hair toxicology. Exemplary off-label and on-label medical drugs are provided in FIG. 7.

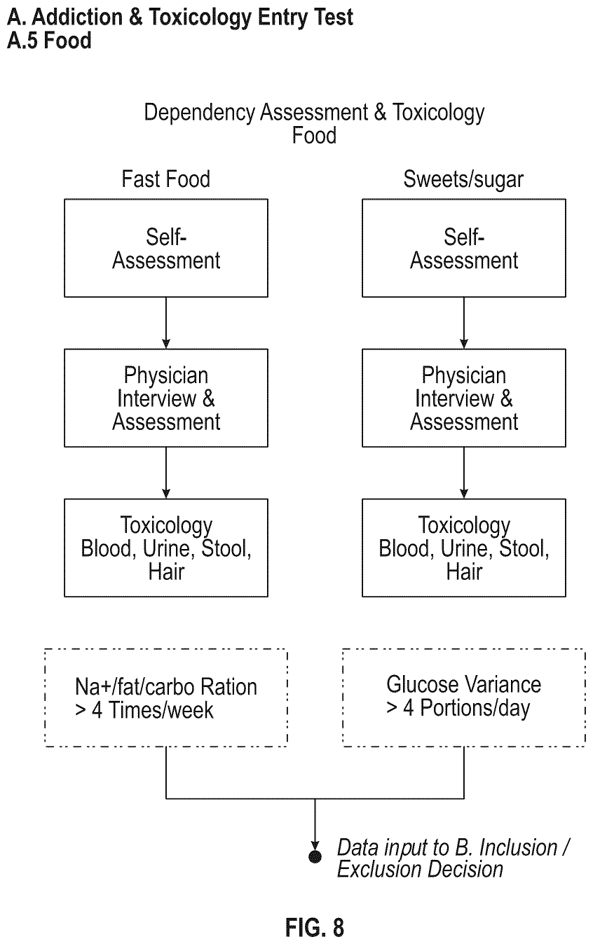

FIG. 8 shows exemplary dependency assessments and toxicology for fast food and sweets/sugar consumption. Assessments can include self-assessment, followed by physician interview and assessment. Following assessments, toxicology may include blood, urine, stool, and/or hair toxicology. Exemplary quantifications for fast food and sweet/sugar consumption are provided in FIG. 8.

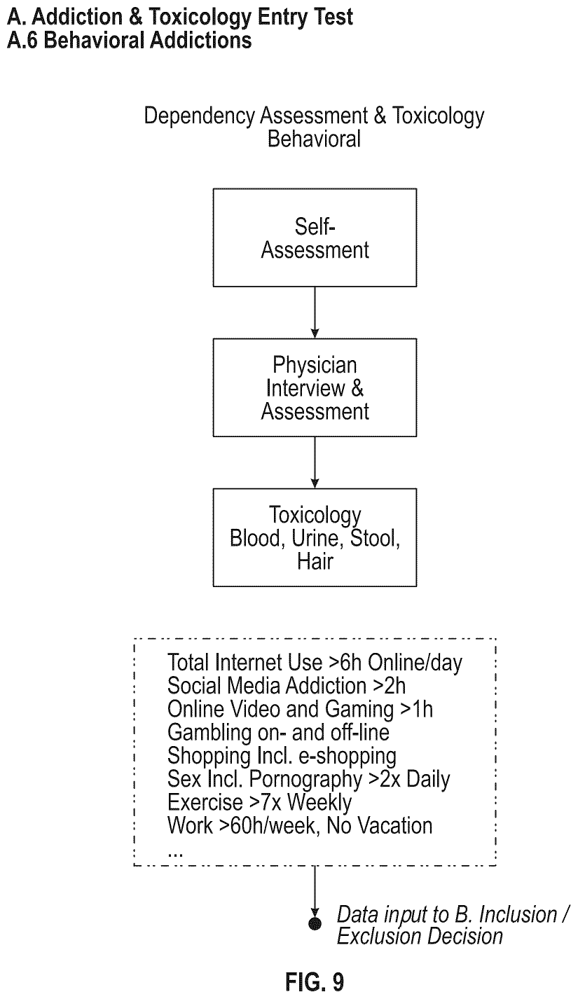

FIG. 9 shows exemplary dependency assessments and toxicology for behavioral addictions. Assessments can include self-assessment, followed by physician interview and assessment. Following assessments, toxicology may include blood, urine, stool, and/or hair toxicology. Exemplary behavioral addictions are provided in FIG. 9.

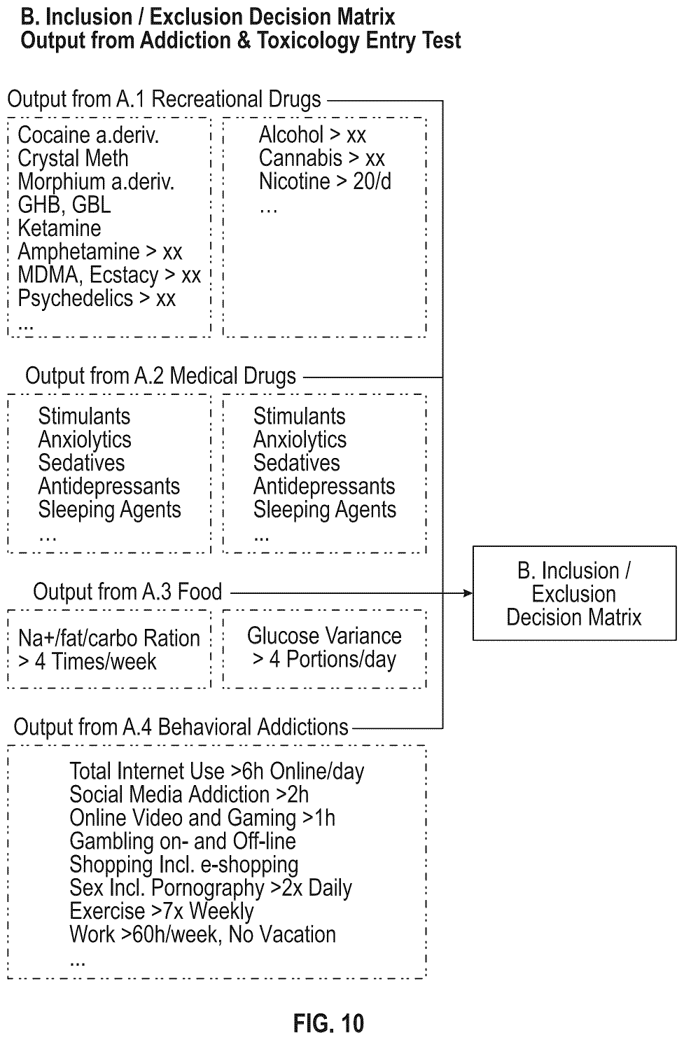

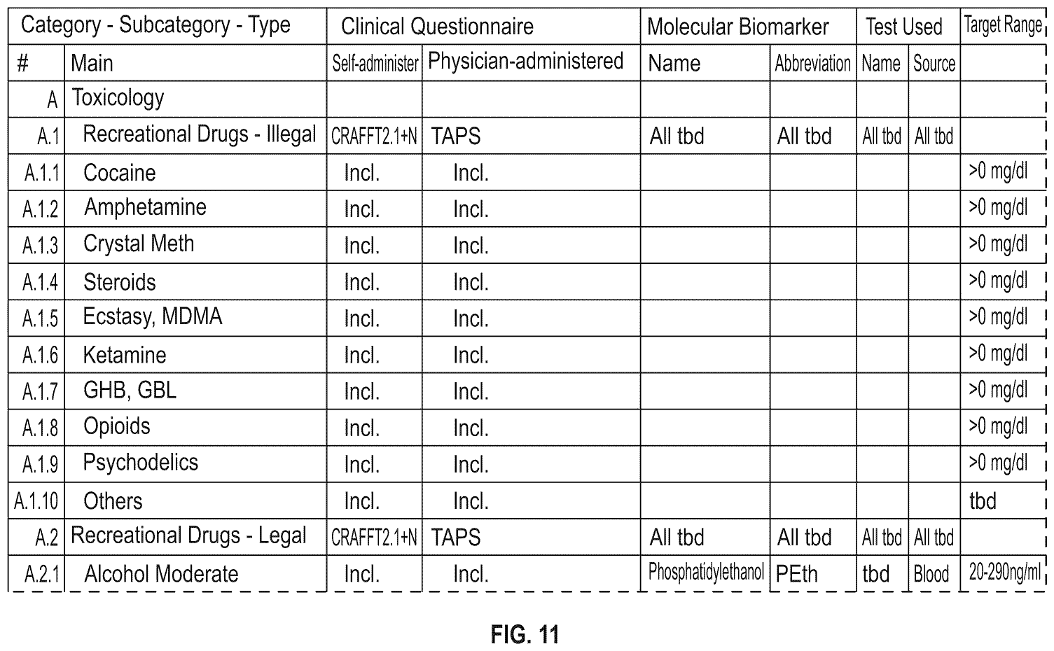

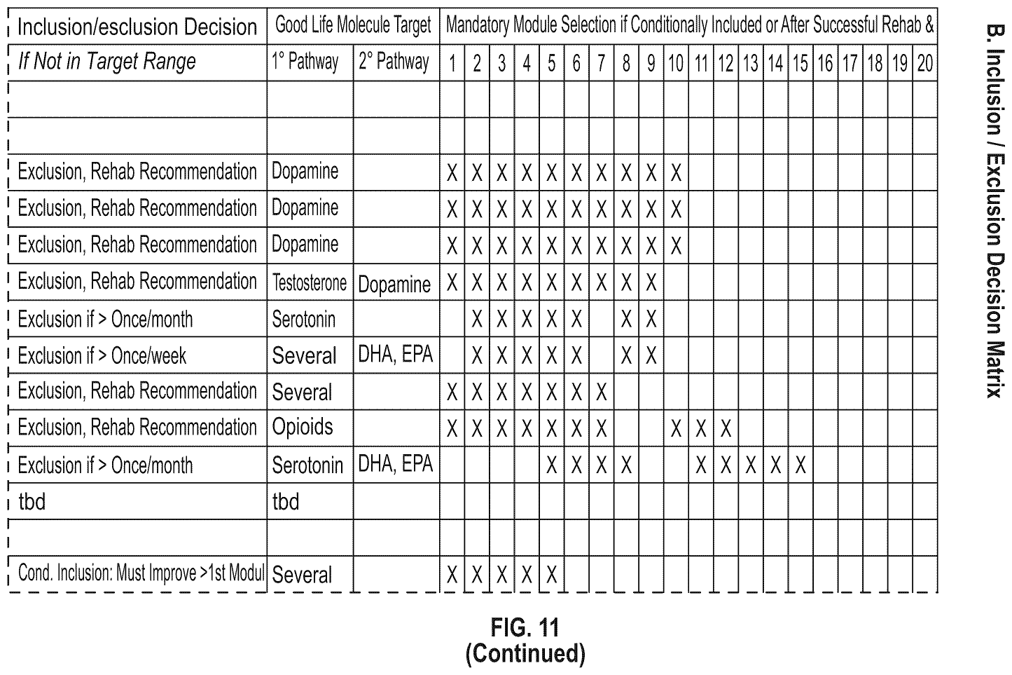

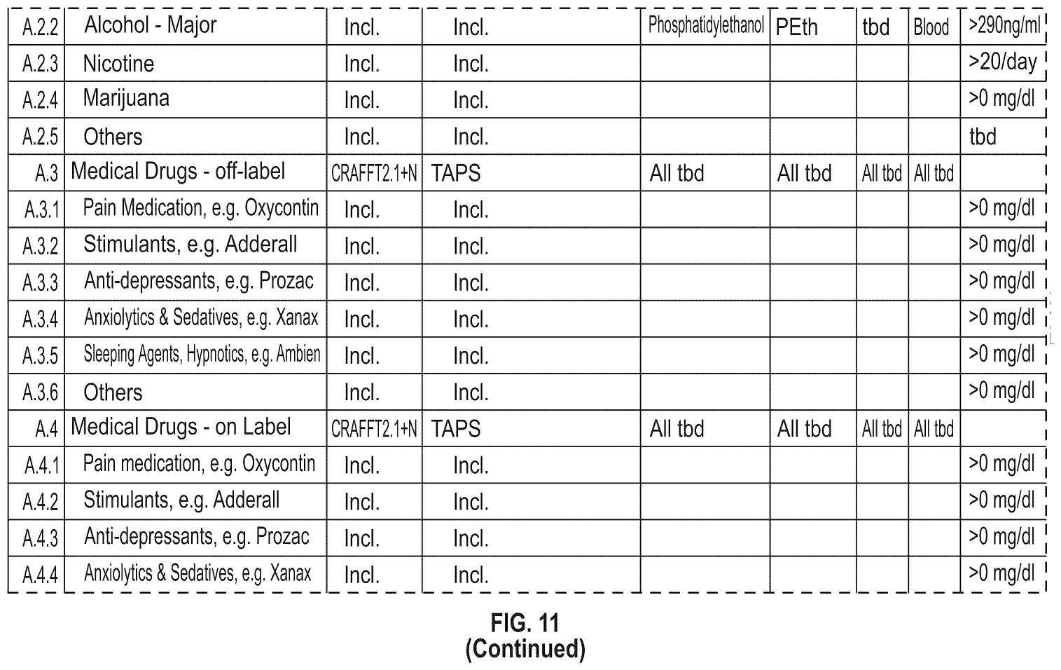

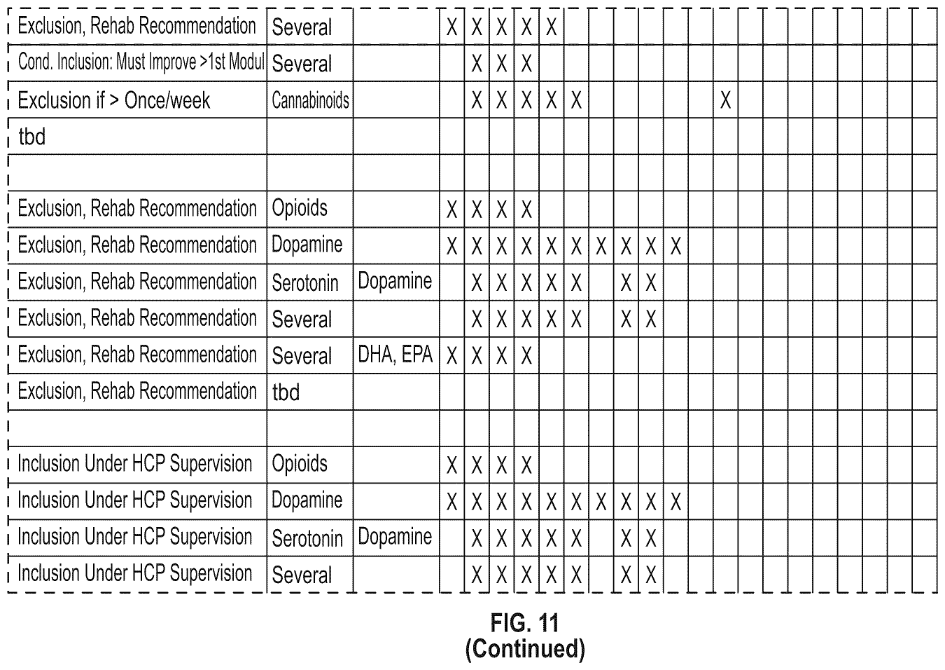

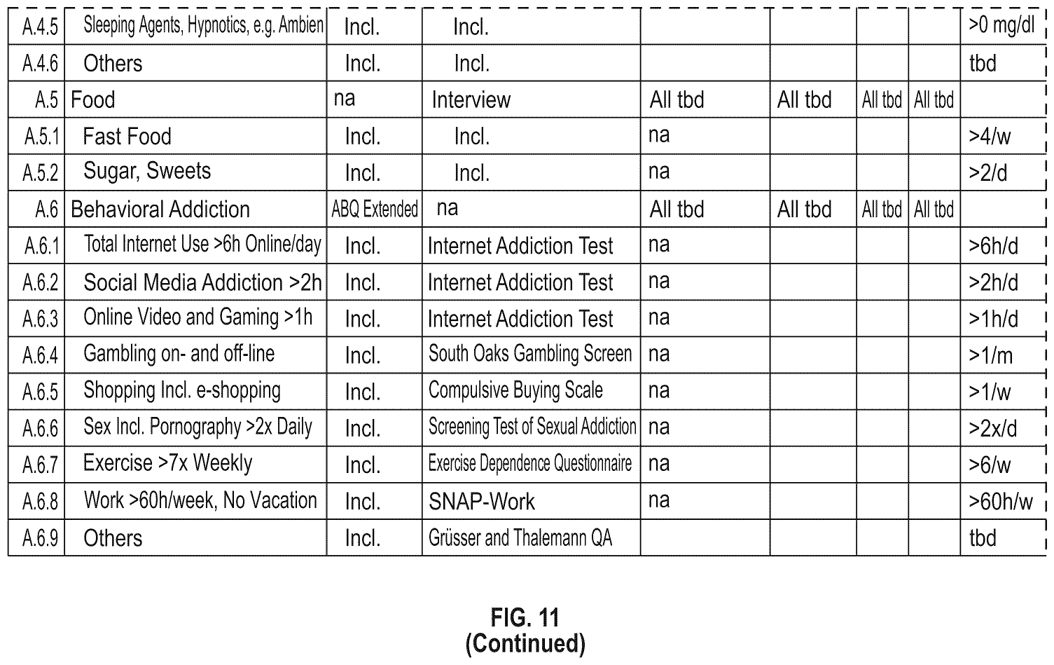

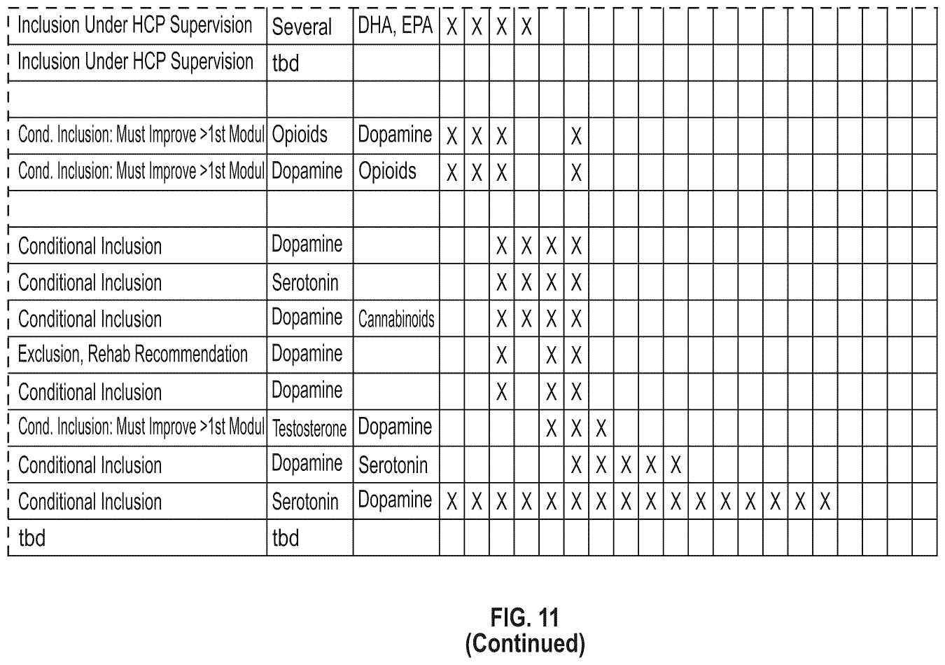

FIG. 10 shows the output data generated by the various addiction and toxicology entry criteria from FIGS. 5-9 as inputs to an inclusion/exclusion decision matrix, which may be automatically executed by an electronic system for determining whether to include a patient in the additional assessments, treatments, and intervention. FIG. 11 shows an exemplary inclusion/exclusion decision matrix, in accordance with some embodiments.

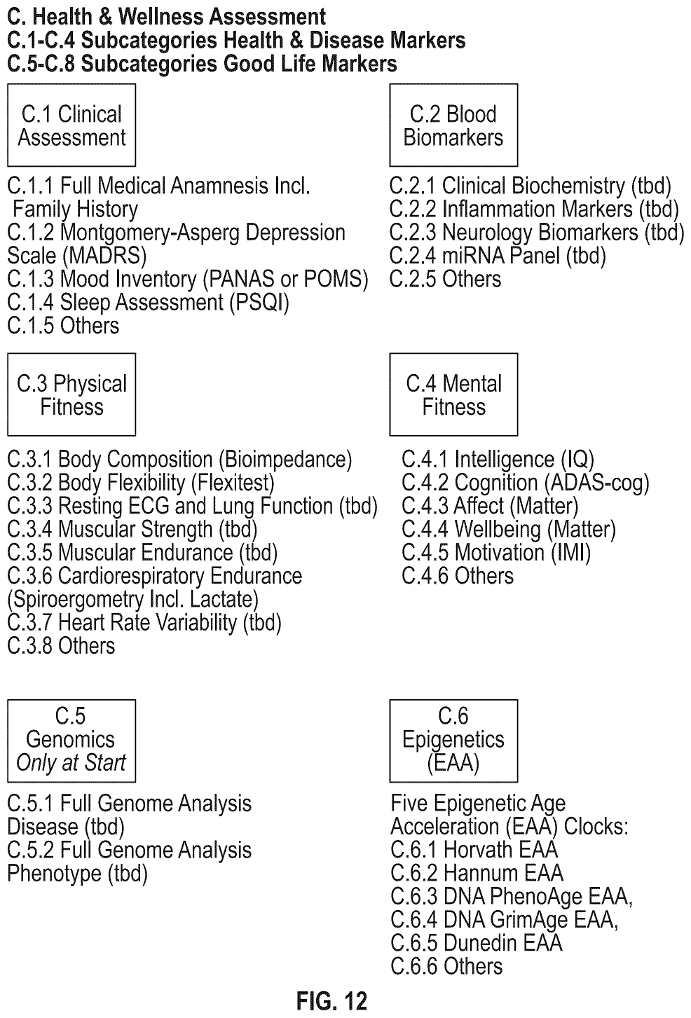

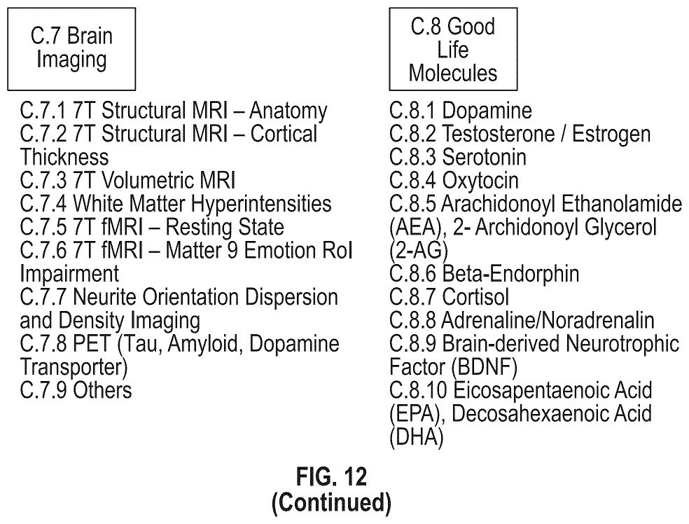

Returning to FIG. 4, if the patient is determined to be eligible from the criteria in FIGS. 5-9 based on application of the matrix in FIGS. 10-11, then the patient may be subjected to a health and wellness assessment at block C. The health and wellness assessment may provide a large set of diagnostic markers from different sources, as described in greater detail above. FIG. 12 shows information regarding the various subcategories of the health and wellness assessment introduced above, including categories for assessing health and disease as well as “good life” wellness markers. Health and disease markers may in some examples include clinical assessment markers (block C1, examples of which are indicated below the block C1 in FIG. 12), blood biomarkers (block C2, examples of which are indicated below the block C2 in the figure), physical fitness markers (block C3, examples of which are indicated below block C3 in the figure), and mental fitness markers (block C4, examples of which are indicated below block C4 in the figure). Good life markers may in some examples include genomics markers (block C5, examples of which are indicated below block C5 in the figure), epigenetics markers (block C6, examples of which are indicated below block C6 in the figure), brain imaging markers (block C5, examples of which are indicated below block C5 in the figure), and the good life molecules (block C8, listed below block C8 in the figure). Health and disease markers and good life markers are also discussed in greater detail above with reference to the knowledge graph, and it is to be understood that any biomarkers described here or with reference to FIG. 12 are applicable to the knowledge graph explanation, and vice versa. These markers can be linked to the set of molecules (e.g., ten molecules or groups of molecules) described above and with reference to block C8, and can be systematically addressed by the interventions.

Following the health and wellness assessment at block C of FIG. 4, an initial molecular good life score may be computed at block I. The initial good life score may be used as a baseline by the AI agent for improving the good life function maximum. The initial good life score may be determined as described herein in greater detail with respect to at least FIGS. 3A-3B.

As described herein, in some examples, if one or more certain diagnostic markers, or categorization of markers, are not in the desired range in the health and wellness assessment, then a corresponding intervention module may be automatically selected for a user by system logic, and an output indicating the selected intervention module (and/or a selected intervention within said module) can be automatically generated by an outputter (e.g., a computer device configured to send a transmission and or display information on a graphical interface). The outputter can be configured to guide the patient through the intervention module (or specific intervention).

In some examples, the outputter may be configured to generate alerts, or notifications, that guide the patient through the intervention module. The outputter may generate alerts that periodically notify the patient of their progress through the intervention module. The outputter may generate alerts that notify the patient when it is time to complete a health and wellness assessment. Alerts may be generated that indicate to the patient that they are escalating to the next intervention, and/or that the patient has successfully completed an intervention.

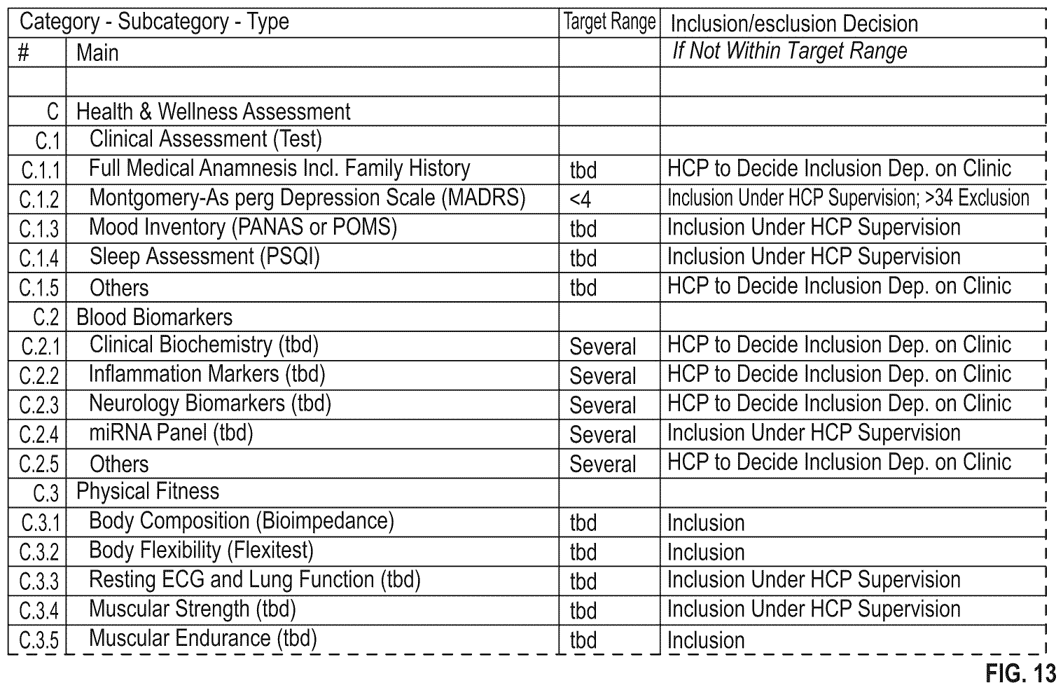

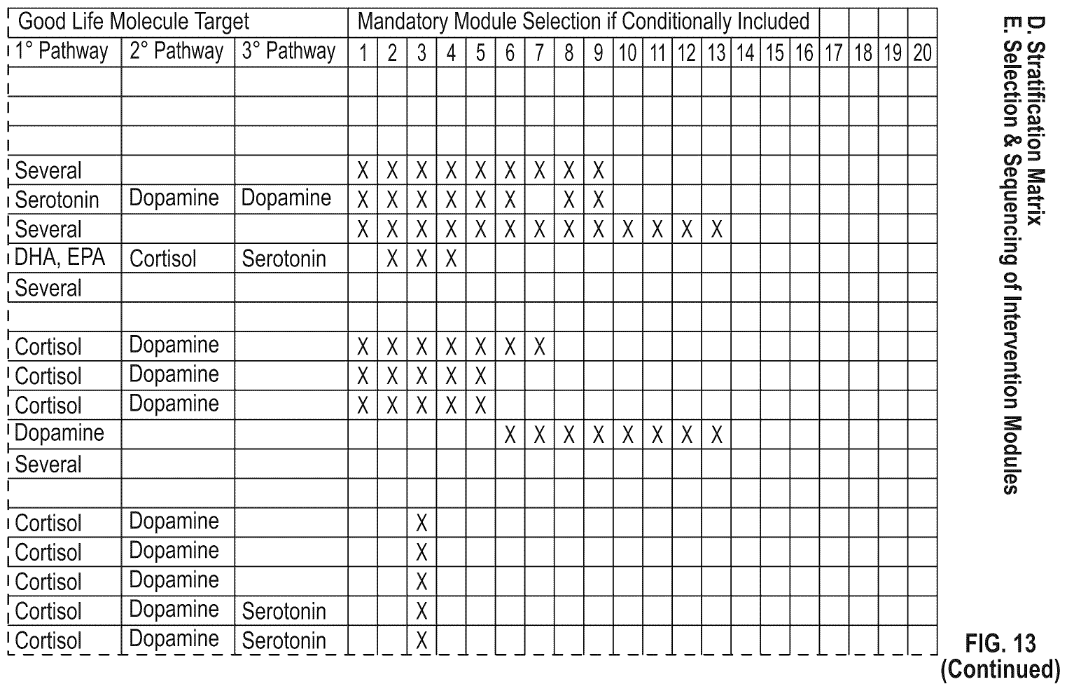

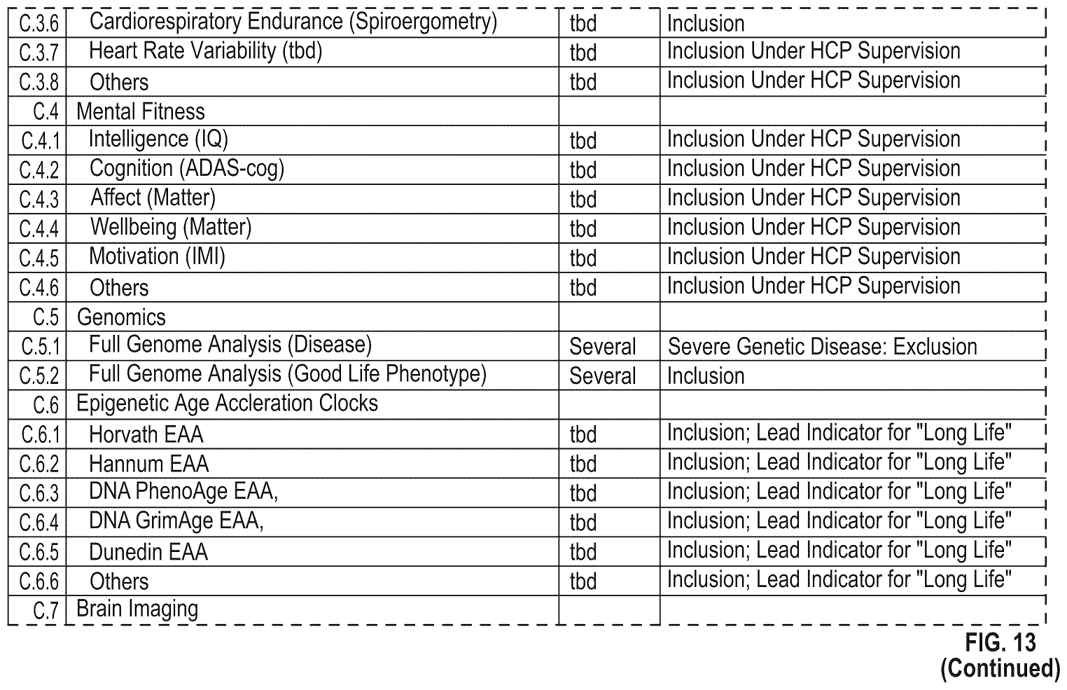

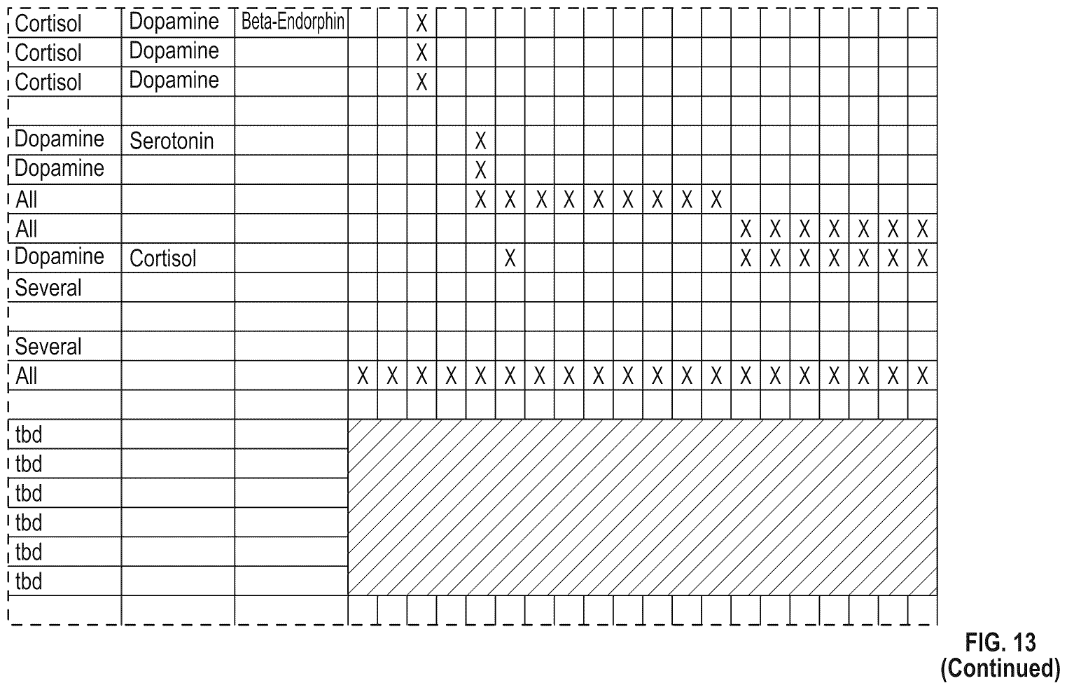

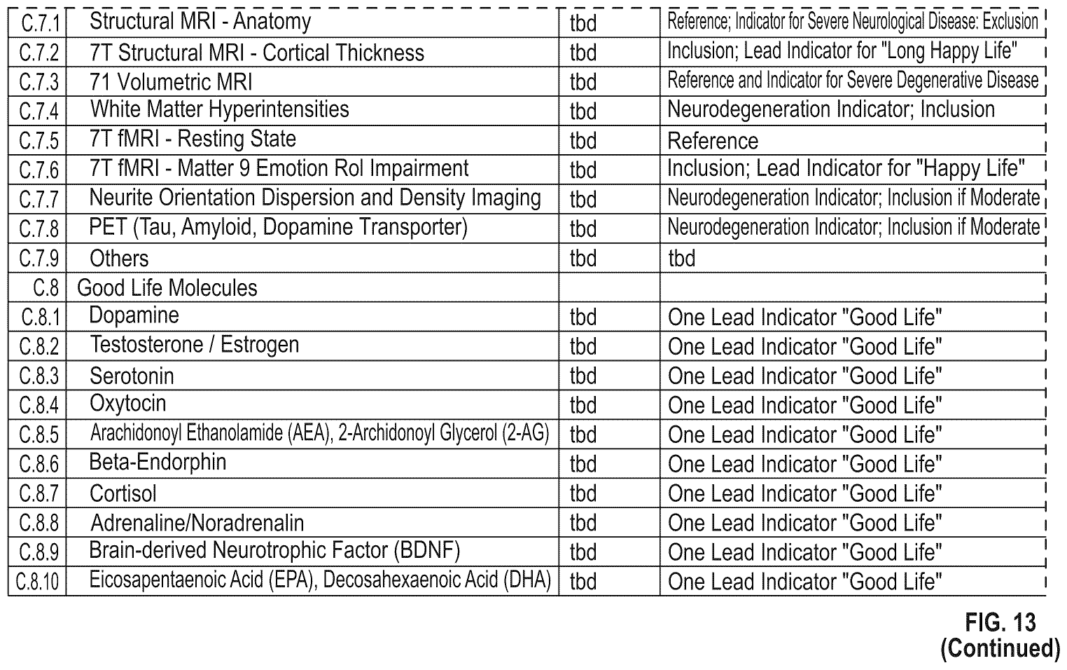

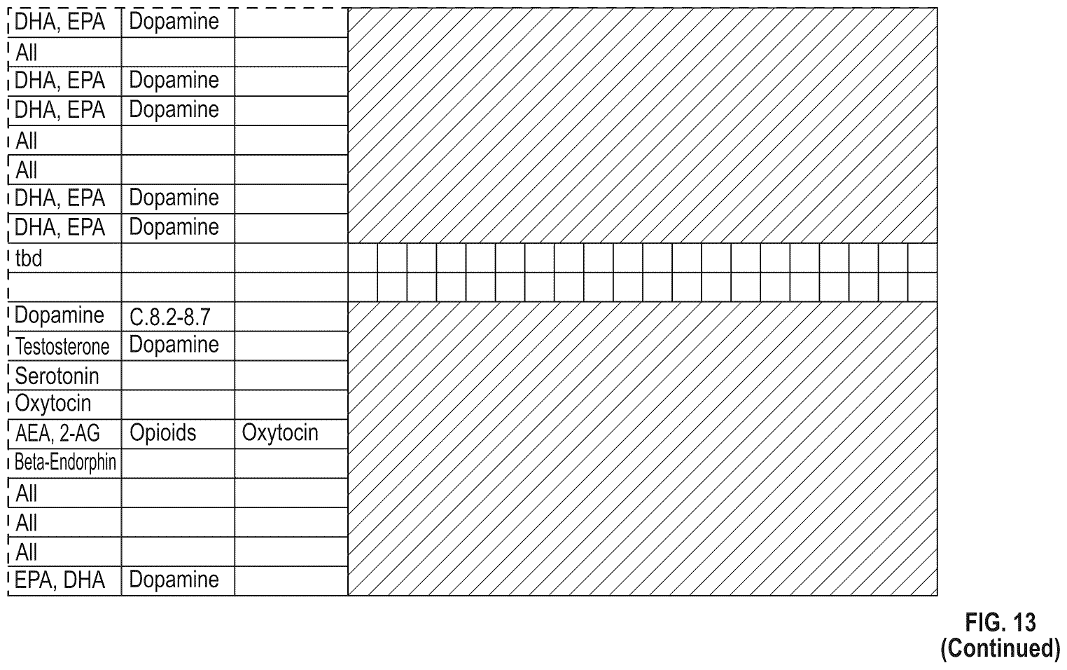

Returning briefly to FIG. 4, following the health and wellness assessment at block C, the method can proceed to a stratification step at block D. FIG. 13 shows an exemplary stratification matrix which may be used for selecting one or more intervention modules (and/or interventions therein), and which may be used to select a sequence of intervention modules (and/or interventions therein) based on the health and wellness assessment data.

In one example use of the stratification matrix, if a patient is already hitting all target ranges for physical fitness and sleep, the stratification matrix may be used to determine that respective modules for physical fitness and sleep may not be needed and thus may not be selected. In some examples, toxicology may be a part of stratification. Toxicology may provide the most direct insight into what is missing from the ten molecules or sets of molecules, because humans tend to substitute their neurochemical gaps with drugs or addictive behaviors. In FIG. 4, the intervention modules are selected at block E, following stratification at block D. As shown in the figure, a machine learning (e.g., AI) model at block K may be utilized to complete each of the modules described herein with respect to blocks C, D, and E (as demonstrated by the connections in the diagram in FIG. 4). The machine learning model can be trained on a knowledge graph, as described herein, to process the biomarker data, stratify, and determine and select one or more intervention modules.

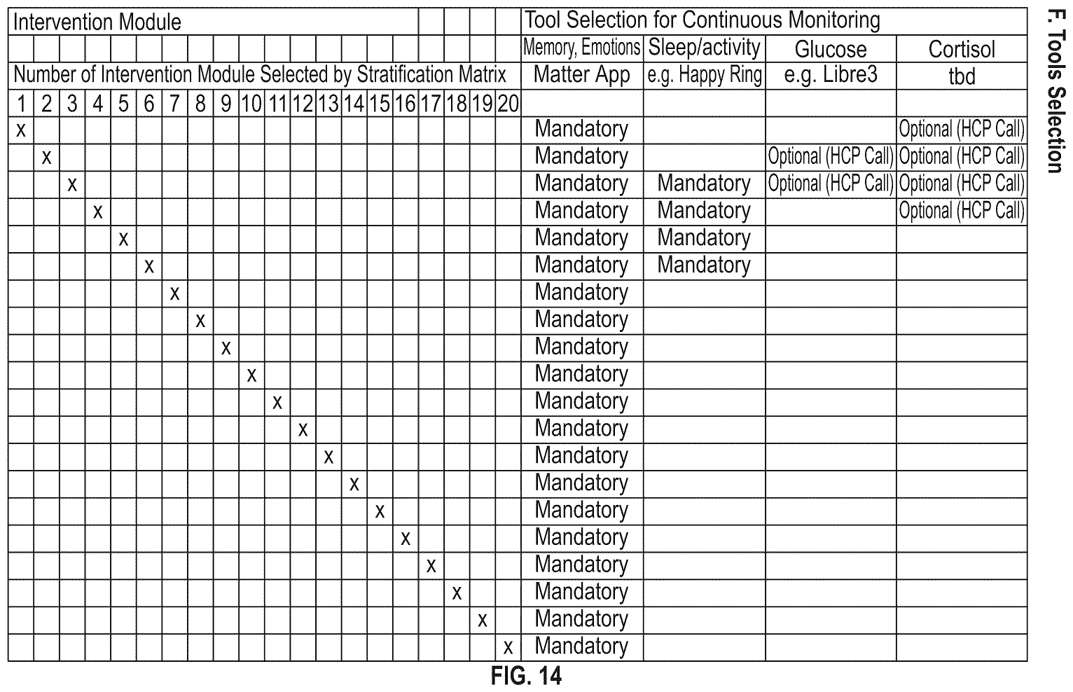

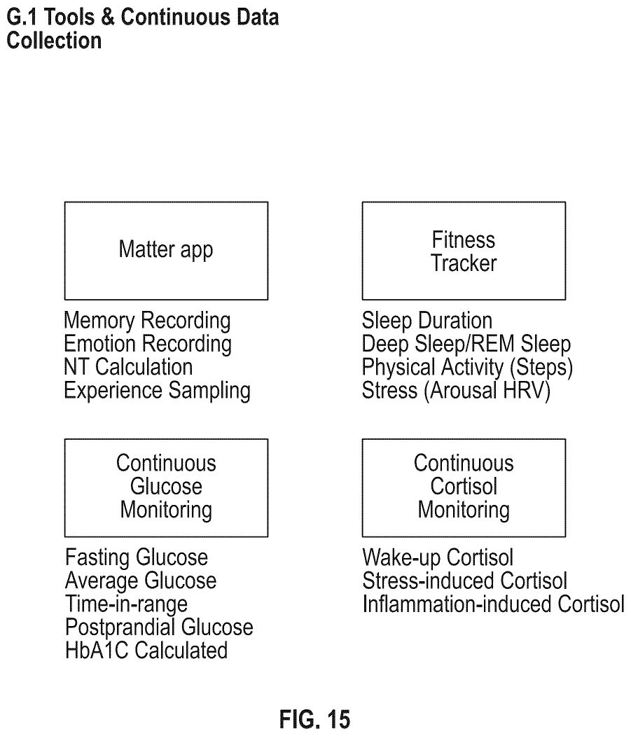

Dependent on the intervention module(s) selected, the method may include selecting one or more continuous tracking tools for the interventions at block F. FIG. 14 shows information regarding tool selection based on the selection of different modules. For example, as shown, the Matter app described herein may be indicated as mandatory for each of the interventions, whereas a wearable monitoring device such as a smart ring may be mandatory for only a portion of the interventions related to sleep/activity tracking. FIG. 15 shows types of data that may be monitored and gathered by the various tools that may be selected. Tools may include, but are not limited to, the Matter app, a fitness tracker, a continuous glucose monitor, and a continuous cortisol monitor.

In some examples, when one or more intervention module(s) are selected by the system, an output can be automatically generated that indicates to the patient which intervention module(s) and which interventions/treatments therein have been selected. The output may be electronically transmitted to a patient, used to update information stored in association with the patient, automatically rendered for display to a patient or caregiver, and/or used to automatically control an operation state of one or more automated devices (e.g., to automatically provide electronic coaching, automatically place a patient in communication with another human being (whether a family member, friend, or medical or mental health professional), and/or automatically administer a pharmacological treatment).

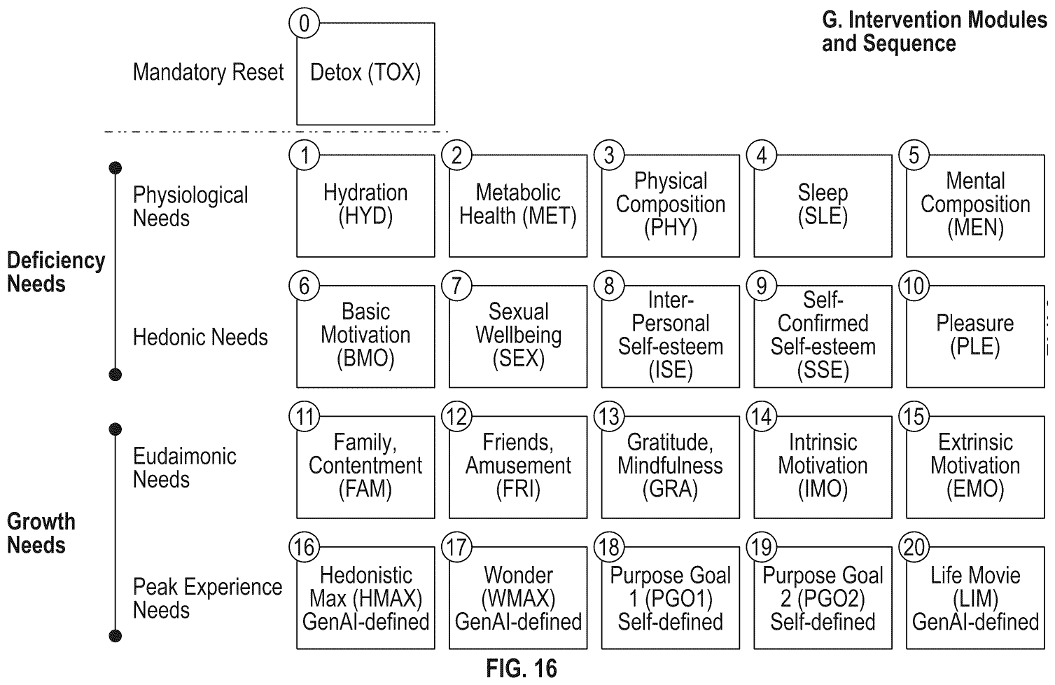

FIG. 16 shows a plurality of intervention modules that may be selected for maximizing the good life function, in accordance with some embodiments. Interventions may fall into modules including but not limited to interventions for improving physiological needs, hedonic needs, eudaimonic needs, and/or peak experience needs. Exemplary interventions within each of these modules are shown in FIG. 16. Interventions may include, but are not limited to, hydration, metabolic health, physiological composition, sleep, mental composition, basic motivation, sexual wellbeing, interpersonal self-esteem, self-confirmed self-esteem, pleasure, family contentment, friends/amusement, gratitude/mindfulness, intrinsic motivation, extrinsic motivation, hedonistic max, wonder, purpose goal 1, purpose goal 2, and/or life movie. In some examples, an intervention can include a mandatory reset, or detox. The machine learning model at block K in FIG. 4 may guide the patient through n selected interventions, or intervention modules (block G in FIG. 4). Guiding the patient through an intervention may include using one or more continuous monitoring tools to gather data as the intervention progresses. In some examples, an AI agent guides (e.g., coaches) the patient through an intervention based on a predefined regimen associated with the intervention. In some examples, an AI agent guides the patient through an intervention based on a specially curated regimen developed by the AI agent based on the patient's biomarker data. In some embodiments, the AI agent dynamically updates the regimen of the intervention based on data received during the intervention that is being continuously monitored.

In some embodiments, the AI agent relies on a knowledge graph, as described herein, to select intervention(s) and guide the patient through the interventions. In some embodiments, the knowledge graph is used to monitor the patient's progress through an intervention. For example, the knowledge graph may ingest continuously monitored data and assess the patient's progress through the intervention based on associations between biomarkers in the knowledge graph.

In some examples, guiding the patient through the intervention includes x rounds of escalations of interventions. In some examples, completing an intervention includes completing n biomarker panels to determine whether a target biomarker range has been achieved. If the target biomarker range has not been achieved, the intervention can be escalated. In some examples, completing an intervention includes completing n additional health and wellness assessments. The additional health and wellness assessments can contribute to the patient's molecular good life score (block J, as shown in FIG. 4).

Once the health and wellness assessment meets the requisite criteria for accomplishing the intervention, subsequent (n+1) interventions may be selected at block H. The above-described intervention module process can be repeated for each of the remaining intervention modules, e.g., in a stepwise fashion as described herein. In some examples, after each intervention, or after each module of interventions, an updated molecular good life score may be determined at block J. The updated good life score may be computed as described above at least with respect to FIGS. 3A-3B. The updated molecular good life score may be used as a new baseline by the AI agent for maximizing the good life function of the patient.

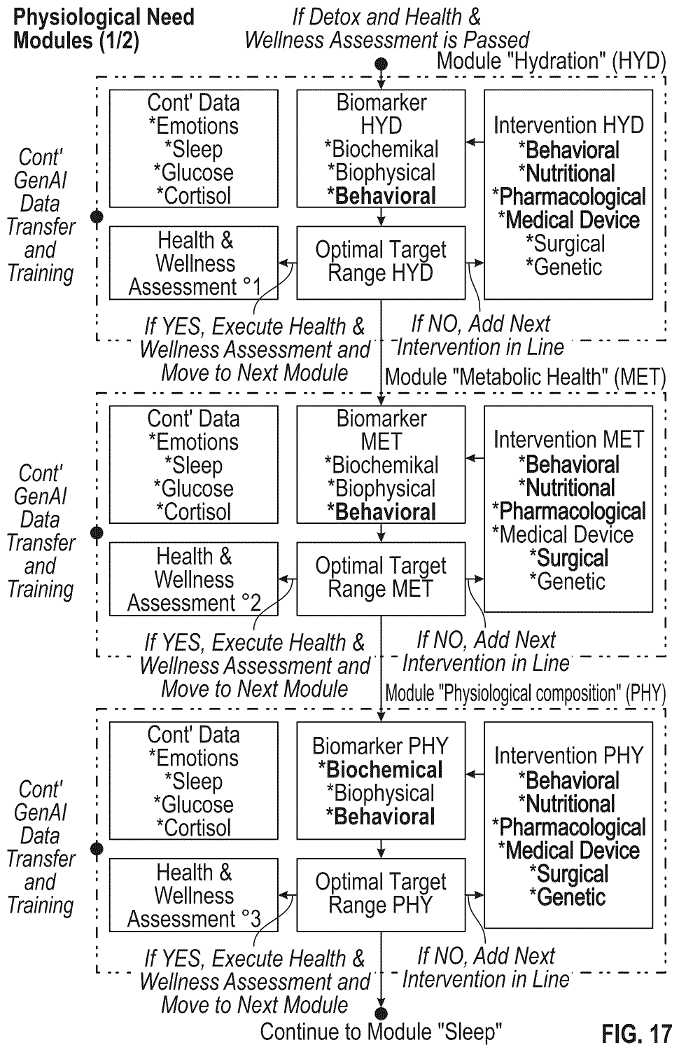

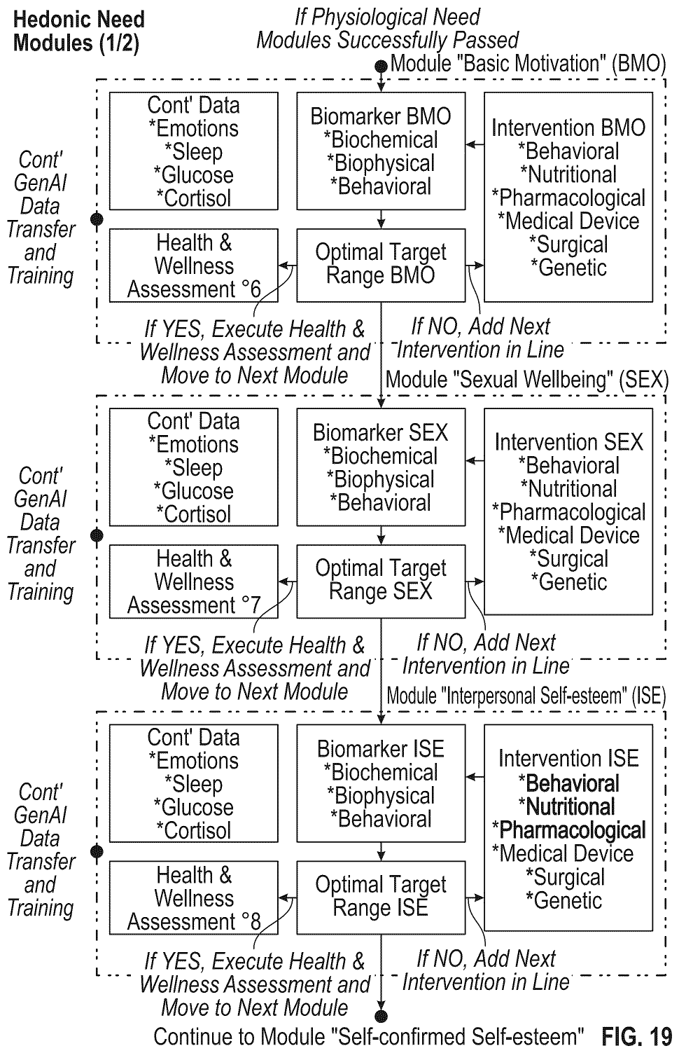

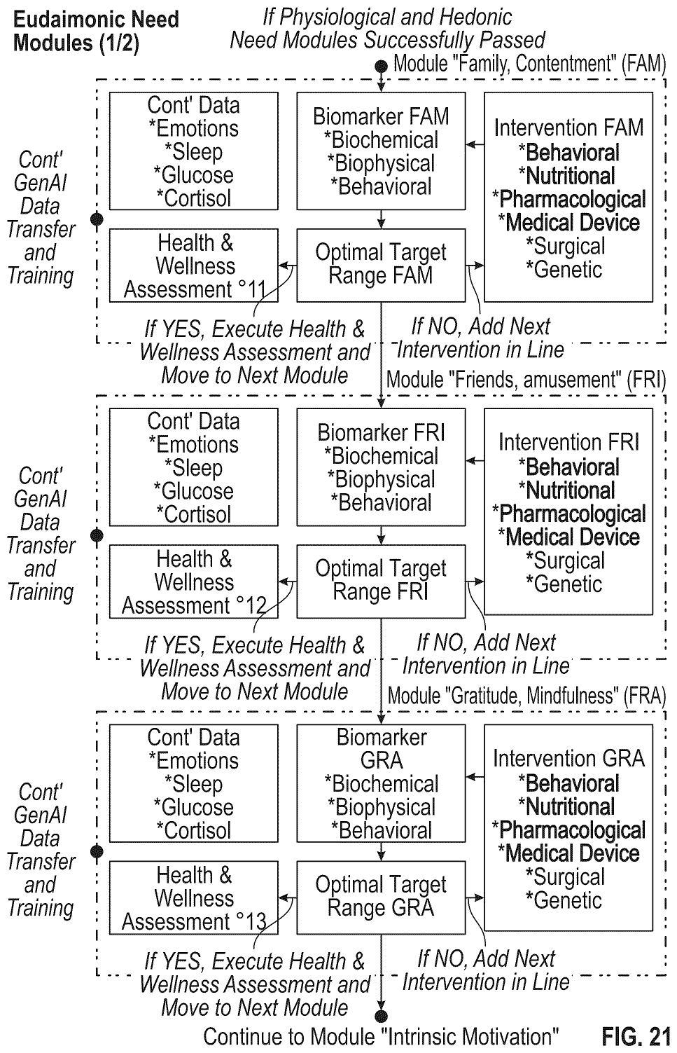

FIGS. 17-22 show an exemplary sequence of intervention modules that may be followed by the AI agent. As described herein, the AI agent may in some examples skip one or more of the intervention modules (or interventions) based on a determination that the patient does not need the intervention. FIG. 17 shows modules for hydration, metabolic health, and physiological composition, in accordance with some embodiments. With reference to the example of the hydration module, the module can include one or more biomarker target ranges for biochemical and/or biophysical. The module can include one or more interventions that are behavioral, nutritional, pharmacological, and/or medical device-related. Continuous monitoring may be performed at the module to determine whether interventions within the module should be escalated, or whether the target ranges have been achieved. Once target ranges have been achieved based on the continuous monitoring data, the health and wellness assessment may be reapplied, and the system may generate an output that causes the patient to be progressed to the next module in the sequence. The intervention module may be AI-based such that captured data is ingested and processed by an AI model to determine whether the patient has achieved the optimal target range for the related biomarkers, and whether the patient can progress to the next module.

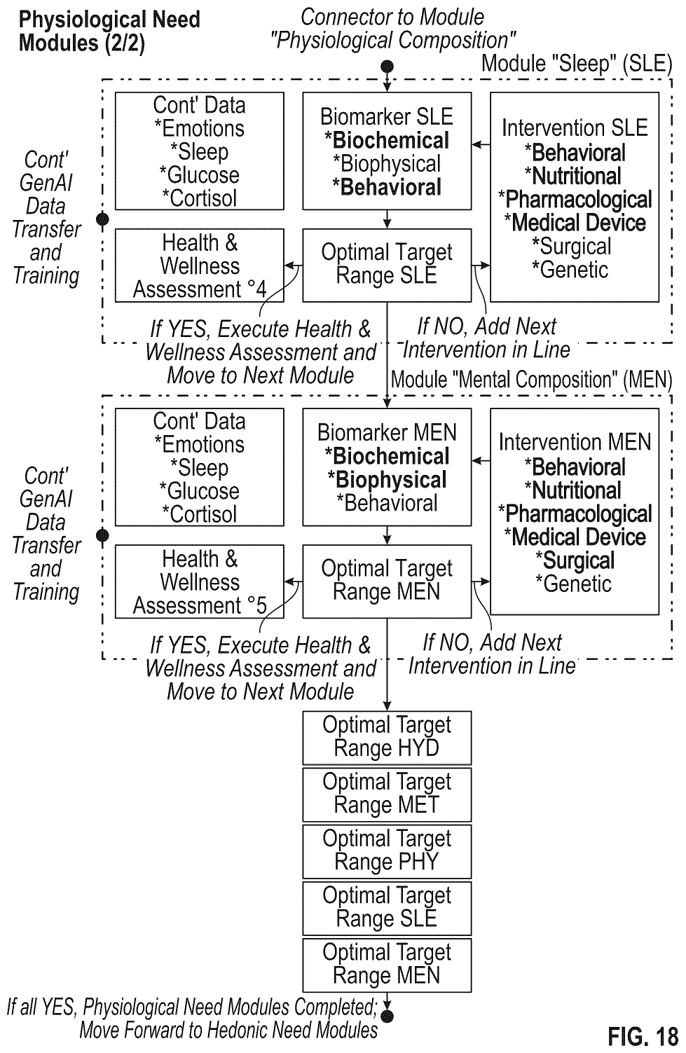

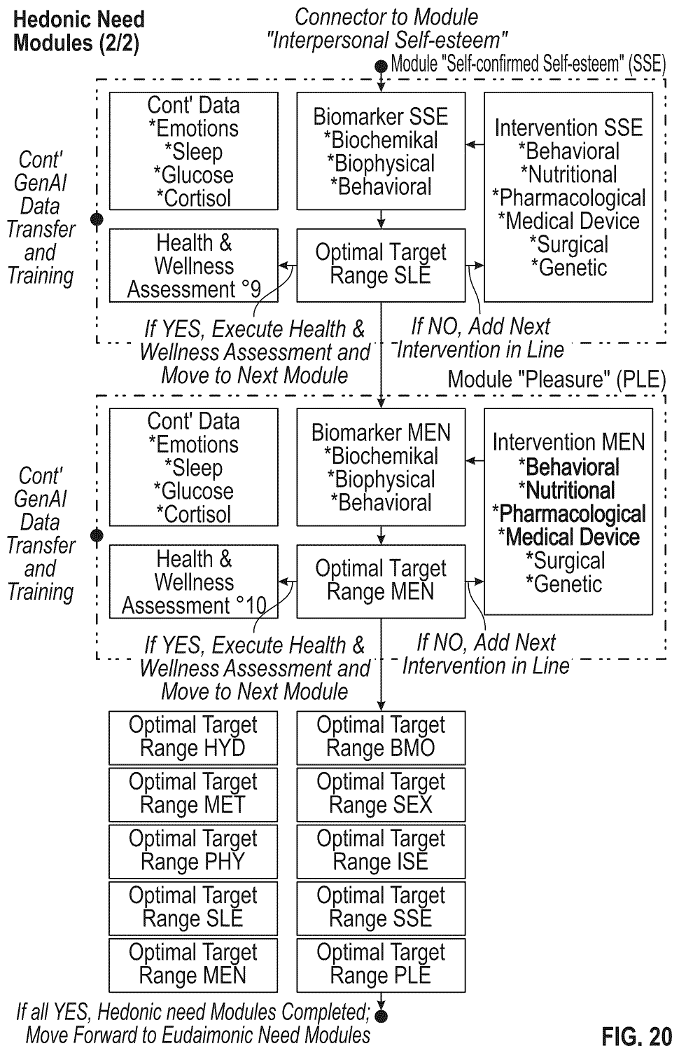

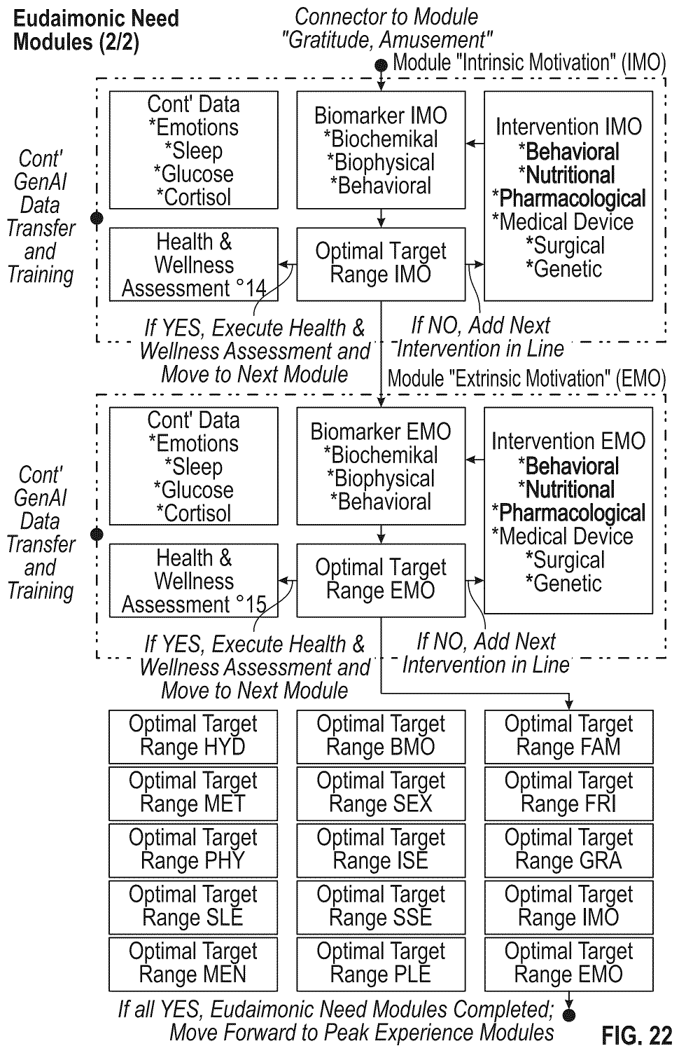

As shown in FIGS. 17-22, the remaining modules within the physiological needs, hedonic needs, and eudaimonic needs categories may progress sequentially in a similar manner as described with respect to the hydration module. In FIG. 18, once the optimal ranges for the physiological need modules (hydration, metabolic health, physical composition, sleep, mental composition) are achieved, the AI agent can progress to the hedonic need modules. In FIG. 20, once the target ranges for each of the hedonic need modules (basic motivation, sexual wellbeing, interpersonal self-esteem, self-confirmed self-esteem, pleasure) are achieved, the AI agent can progress to the eudaimonic need modules. In FIG. 22, once the target range for the eudaimonic need modules (family/contentment, friends/amusement, gratitude/mindfulness, intrinsic motivation, extrinsic motivation) are achieved, the AI agent can progress toward peak experience modules. As shown in FIG. 16, the five modules hedonistic max, wonder, purpose goal 1, purpose goal 2, and life movie, as grouped in the peak experience needs category, may be defined by generative AI and/or self-defined by a patient. The respective biomarker(s) behind a peak experience recommendation may include emotional biomarkers (e.g., absence of high-emotion ratings in memory recordings, high ratings in “boredom”) and/or diagnostic imaging biomarkers (e.g., reduced cortical thickness in key regions, inferior brain activation speed, maximum intensity in key regions representing positive emotions).

The selected interventions may be administered, for example by the patient, a medical practitioner, and/or by automated device (e.g., executing an AI agent). During administration of the interventions, continuous monitoring by the selected tools may be performed. As described herein, if target levels for biomarkers associated with the module are not achieved, then an escalating intervention within the module may be selected. If target levels for biomarkers associated with the module are achieved, then the system may proceed to the next module, and may assess biomarkers and determine interventions/treatments for that module. In some examples, progressions through the modules may be linear and additive. For example, after successful completion of a first selected module (e.g., the hydration module), a second module 2 (e.g., metabolic health) can be started, and so on. However, while moving on to subsequent modules, the patient may be continuously monitored to ensure that target biomarker ranges of the previously successfully completed modules are maintained. In this way, once all selected interventions are accomplished, target levels for all biomarkers will have been achieved.

As described herein, certain modules may be skipped if the user has already reached the respective biomarker target range associated with this module. In some examples, the system may be configured to progress through increasingly elevated interventions to achieve a target level for a given molecule. For example, drinking 2 L of water a day can reduce your Cortisol level by 25-30%. Cortisol is the most powerful antagonist to other molecules listed herein. Thus, the system may be configured such that, based on a determination that the patient's cortisol needs to be lowered, it recommends starting with a simple intervention such as drinking water, instead of a more invasive intervention like exercise five times weekly (which also lowers Cortisol by 25-30%), or taking heavy sleeping pills, or taking antidepressants that counteract the Cortisol only indirectly.

A patient may be guided through the program by an automated coach, by a professional (e.g., physician) who is consulting the outputs of the system, or may be self-guided in a similar fashion (i.e., by interpreting the outputs of the system). For example, every 3-6 months, the patient may visit a physical clinic to get a next health & wellness assessment, and the user may continuously access intervention recommendations provided by their GenAI coach. As described herein, the intervention modules can be additive, e.g., the hydration module can address a distinct subset of biomarkers associated with hydration. Once a target range for the biomarkers is reached, a second module (e.g., metabolic health) can begin to address another distinct subset of biomarkers associated with the second module. The biomarkers associated with the first module can be carried forward throughout the progression of interventions and can be monitored on a regular basis during the treatment plan. In some examples, the biomarkers associated with previous modules can be monitored at the beginning of a subsequent module, using the health & wellbeing assessment described herein.

In this manner, the system may thus progress a patient through all the relevant modules, using continuous monitoring to ensure that biomarkers for all previously completed modules remain within target ranges and dynamically outputting and/or controlling administration of interventions/treatments if they are not. The patient may eventually progress through all the modules, maximizing the GL function to within target ranges for each of the modules.

### Systematic Machine Learning Support—Personalized Generative Artificial Intelligence for Determining Interventions to Maximize Quantification of Health and Wellbeing

In some embodiments, one or more artificial intelligence (AI) models (e.g., LLM's or the like) may be trained based on data representing a large, multi-modal set of biomarkers systematically monitored over several rounds of different interventions. The AI models may use a knowledge graph or similar structure of associations between biomarkers to train the AI based on the data. The data may be specific to a patient, and may be used as training data for refining the AI model to be curated for the specific patient. In this way, the AI model can provide unique recommendations and/or other outputs usable by the patient to determine and select interventions/treatments that increase their health and wellness and maximize a quantification of health and wellness as described herein.

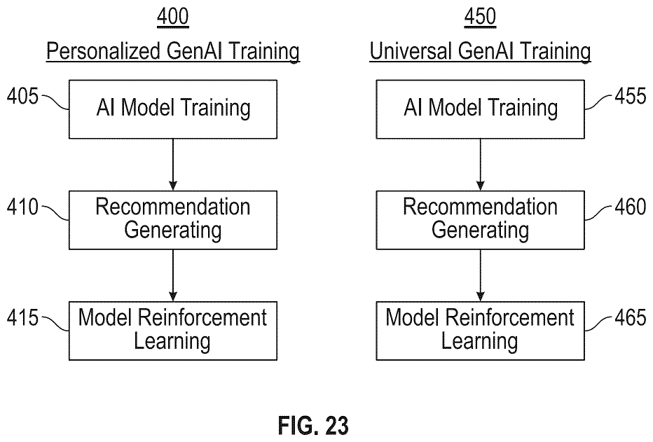

FIG. 23 shows a process 400 for training, deploying, and refining a personalized AI model, according to some embodiments. The process 400 can include training the model (block 405), followed by generating recommendations (block 410), followed by model reinforcement learning (block 415). In this way, the personalized AI models can be iteratively trained and updated based on learnings from a specific patient's biomarker data.

At block 405, training the model can include receiving a pre-trained language model (or other similar AI model), receiving user-specific training data (e.g., brain scan data, positive memory data, self-reported emotional experience data, and/or data regarding outcomes from prior interventions performed in accordance with a module scheme as described herein), and applying supervised training that modifies the pre-trained model based on the user-specific training data and based on known associations and relationships between variables of the GL function (e.g., nodes in the knowledge graph).

At block 410, generating recommendations can include applying the specialized user-specific model to generate intervention recommendations for the patient based on unmet needs of the patient and to improve the patient's health and wellbeing by maximizing the GL function (in a similar manner as described above).

At block 415, reinforcement learning for the model(s) can include applying recursive training updates to the user-specific model based on outcomes of the executed interventions.

### Systematic Machine Learning Support—Universal Generative Artificial Intelligence for Determining Interventions to Maximize Quantification of Health and Wellbeing

In some embodiments, one or more artificial intelligence (AI) models (e.g., LLM's or the like) may be trained based on data representing a large, multi-modal set of biomarkers systematically monitored over several rounds of different interventions. The AI models may use a knowledge graph or similar structure of associations between biomarkers to train the AI based on the data. The data may be data for a population of different individuals (e.g., 1000 or more individuals) that can be used as training data for refining universal AI models, i.e., those that may not be user-specific. In this way, the universal AI agent can provide recommendations and/or other outputs usable by patients based on a larger, patient-agnostic data set to determine and select interventions/treatments that increase their health and wellness and to maximize a quantification of health and wellness as described herein.

As shown in FIG. 23, a process 450 for training, deploying, and refining universal AI model can include training the model (block 455), followed by generating recommendations (block 460), followed by model reinforcement learning (block 460). In this way, the AI models can be iteratively trained and updated based on learnings from a specific patient's biomarker data.

At block 455, training the model can include receiving a pre-trained language model (or other similar AI model), receiving non-user-specific training data (e.g., brain scan data, positive memory data, self-reported emotional experience data, and/or data regarding outcomes from prior interventions performed in accordance with a module scheme as described herein), and applying supervised training that modifies the pre-trained model based on non-user-specific training data and based on known associations and relationships between variables of the GL function (e.g., nodes in the knowledge graph).

At block 460, generating recommendations can include apply the specialized non-user-specific model to generate intervention recommendations for a specific patient based on unmet needs of the patient to improve the patient's health and wellbeing by maximizing GL function.

At block 465, reinforcement learning for the AI model(s) can include applying recursive training updates to the non-user-specific model based on outcomes of the executed interventions.

In some examples, the non-user-specific ML training process 450 may be used in parallel and/or in series with the user-specific ML training process 400 shown in FIG. 23.

### Computing Systems

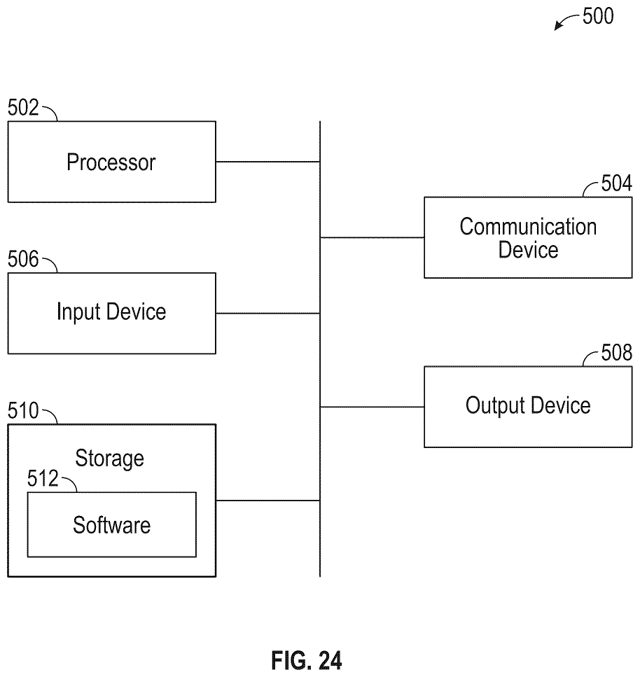

FIG. 24 shows an exemplary computer system 500 that can be used to execute the methods for determining interventions/treatments that maximize health and wellbeing as described herein. System 500 can be any suitable type of processor-based system, such as a personal computer, workstation, server, handheld computing device (portable electronic device) such as a phone or tablet, or dedicated device. System 500 may be configured to execute software causing it to perform all or part of any of the methods described herein. The system 500 can include, for example, one or more of input device 520, output device 530, one or more processors 510, storage 540, and communication device 560. Input device 520 and output device 530 can generally correspond to those described above and can either be connectable or integrated with the computer.

Input device 520 can be any suitable device that provides input, such as a push-button switch, a touch screen, keyboard or keypad, mouse, gesture recognition component of a virtual/augmented reality system, or voice-recognition device. In some examples, input device 520 is a wearable sensing device (e.g., a smart ring, watch, band, or similar), or a biomarker monitoring device (e.g., glucose monitoring device, cortisol monitoring device, etc.). Output device 530 can be or include any suitable device that provides output, such as a display, touch screen, haptics device, virtual/augmented reality display, or speaker. In some examples, output device 530 is a virtual reality (VR) device, such as a VR headset or glasses.

Storage 540 can be any suitable device that provides storage, such as an electrical, magnetic, or optical memory including a RAM, cache, hard drive, removable storage disk, or other non-transitory computer readable medium. Communication device 560 can include any suitable device capable of transmitting and receiving signals over a network, such as a network interface chip or device. The components of the computing system 500 can be connected in any suitable manner, such as via a physical bus or wirelessly.

Processor(s) 510 can be any suitable processor or combination of processors, including any of, or any combination of, a central processing unit (CPU), graphics processing unit (GPU), field programmable gate array (FPGA), programmable system on chip (PSOC), and application-specific integrated circuit (ASIC). Software 550, which can be stored in storage 540 and executed by one or more processors 510, can include, for example, the programming that embodies the functionality or portions of the functionality of the present systems and methods.

Software 550 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 540, that can contain or store programming for use by or in connection with an instruction execution system, apparatus, or device.

Software 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport computer readable medium can include, but is not limited to, an electronic, magnetic, optical, electromagnetic, or infrared wired or wireless propagation medium.

System 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 500 can implement any operating system suitable for operating on the network. Software 550 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service.

### CLAUSES

The following clauses are exemplary and are not intended to limit the scope of the disclosure provided herein.

Clause 1. A method to improve and maintain molecular biomarker levels in a patient, comprising:
obtaining data representing a plurality of biomarker levels for a plurality of different molecular biomarkers for a patient;
determining, based on the obtained data representing the plurality of biomarker levels, that the biomarker levels for the patient fall do not fall within one or more target ranges associated with a plurality of respective intervention modules;
selecting, based on the obtained data and using a knowledge graph, a first one of the plurality of respective intervention modules;
selecting a first intervention associated with the selected first intervention module; and
generating, by an outputter, an output indicating the selected first intervention.

Clause 2. The method of clause 1, comprising:
selecting, based on the selected intervention module, one or more continuous monitoring tools from a set of continuous monitoring tools;
obtaining data, via the one or more continuous monitoring tools, indicative of the changes in biomarker levels of the patient in response to the selected first intervention.

Clause 3. The method of clause 2, comprising:
determining, after the changes in biomarker levels, that the changed biomarker levels of the patient fall within the one or more target ranges;
in response to the determination that the changed biomarker levels of the patient fall within the one or more target ranges, generating, by the outputter, an output indicating a subsequent second intervention module and a selected second intervention therein.

Clause 4. The method of clause 2, comprising:
determining, after the changes in biomarker levels, that the changed biomarker levels of the patient do not fall within the one or more target ranges;
in response to the determination that the changed biomarker levels of the patient do not fall within the one or more target ranges, generating, by the outputter, an output indicating an escalated intervention within the first intervention module.

Clause 5. The method of any one of clauses 2-4, wherein the one or more continuous monitoring tools comprise one or more tools selected from:
a survey and/or automated GUI-based data collection tool;
a wearable device for monitoring blood glucose levels;
a wearable device for monitoring cortisol levels; and
a wearable device for monitoring sleep quality, physical activity levels, and/or stress levels.

Clause 6. The method of any one of clauses 1-5, wherein the plurality of different molecular biomarkers for the patient include one or more biomarkers selected from:
Dopamine;
Testosterone and/or Estrogen (which in some embodiments may be referred to and/or quantified as a testosterone level);
Serotonin;
Oxytocin;
Arachidonoyl ethanolamine (AEA) and/or 2-archidonoyl glycerol (2-AG);
beta-Endorphin;
Cortisol;
Adrenaline and/or Noradrenalin;
Brain-derived neurotrophic factor (BDNF); and
Eicosatetraenoic acid (EPA) and/or Docosahexaenoic acid (DHA).

Clause 7. The method of any one of clauses 1-6, wherein selecting the first intervention module and selecting a first intervention therein is performed based on an AI inference model.

Clause 8. The method of any one of clauses 1-7, comprising administering the selected first intervention as a treatment on the patient.

Clause 9. A system comprising one or more processors and memory storing instructions configured to be executed by the one or more processors to cause the system to:
obtain data representing a plurality of biomarker levels for a plurality of different molecular biomarkers for a patient;
determine, based on the obtained data representing the plurality of biomarker levels, that the biomarker levels for the patient fall do not fall within one or more target ranges associated with a plurality of respective intervention modules;
select, based on the obtained data and using a knowledge graph, a first one of the plurality of respective intervention modules;
select a first intervention associated with the selected first intervention module; and
generate, by an outputter, an output indicating the selected first intervention.

Clause 10. A non-transitory computer-readable storage medium storing instructions which, when executed by a system comprising one or more processors, cause the system to:
obtain data representing a plurality of biomarker levels for a plurality of different molecular biomarkers for a patient;
determine, based on the obtained data representing the plurality of biomarker levels, that the biomarker levels for the patient fall do not fall within one or more target ranges associated with a plurality of respective intervention modules;
select, based on the obtained data and using a knowledge graph, a first one of the plurality of respective intervention modules;
select a first intervention associated with the selected first intervention module; and
generate, by an outputter, an output indicating the selected first intervention.

### ADDITIONAL CLAUSES

The following additional clauses are exemplary and are not intended to limit the scope of the disclosure provided herein.

Clause 1. A method to improve and maintain molecular biomarker levels in a patient, comprising: obtaining biomarker data representing a plurality of biomarker levels for a plurality of different molecules for a patient; determining, based on the obtained data representing the plurality of biomarker levels and using a knowledge graph linking one or more intervention modules, one or more target biomarker ranges, and one or more outcomes, that at least one of the plurality of biomarker levels for the patient do not fall within the one or more target biomarker ranges; selecting, based on the obtained data and using the knowledge graph, a first one of the plurality of respective intervention modules associated with the at least one biomarker level that does not fall within the target biomarker range; selecting a first intervention associated with the selected first intervention module; and generating, by an outputter, an output indicating the selected first intervention.

Clause 2. The method of clause 1, comprising: selecting, based on the selected intervention module, one or more continuous monitoring tools from a set of continuous monitoring tools; and obtaining updated biomarker data, via the one or more continuous monitoring tools, indicative of the changes in biomarker levels of the patient in response to the selected first intervention.

Clause 3. The method of clause 2, comprising: determining, after the changes in biomarker levels, that the changed biomarker levels of the patient fall within the one or more target ranges; and in response to the determination that the changed biomarker levels of the patient fall within the one or more target ranges, generating, by the outputter, a second output indicating a subsequent second intervention module and a selected second intervention therein.

Clause 4. The method of clause 2, comprising: determining, after the changes in biomarker levels, that the changed biomarker levels of the patient do not fall within the one or more target ranges; and in response to the determination that the changed biomarker levels of the patient do not fall within the one or more target ranges, generating, by the outputter, a third output indicating an escalated intervention within the first intervention module.

Clause 5. The method of any one of clauses 2-4, wherein the one or more continuous monitoring tools comprise one or more tools selected from: a survey and/or automated GUI-based data collection tool; a wearable device for monitoring blood glucose levels; a wearable device for monitoring cortisol levels; a wearable device for monitoring sleep quality, physical activity levels, stress levels, and/or emotional states correlating with high arousal states; and a wearable device for monitoring movement data, elevation data, and/or co-location or proximity data with other persons.

Clause 6. The method of any one of clauses 1-5, wherein the plurality of different molecules for the patient include one or more molecules selected from: Dopamine; Testosterone and/or Estrogen (which in some embodiments may be referred to and/or quantified as a testosterone level); Serotonin; Oxytocin; Arachidonoyl ethanolamine (AEA) and/or 2-archidonoyl glycerol (2-AG); beta-Endorphin; Cortisol; Adrenaline and/or Noradrenalin; Brain-derived neurotrophic factor (BDNF); and Eicosatetraenoic acid (EPA) and/or Docosahexaenoic acid (DHA).

Clause 7. The method of any one of clauses 1-6, wherein the plurality of different molecules for the patient include at least one of: one or more molecules involved in mitochondrial energy metabolism; one or more molecules involved in neuronal activation, enhanced cognition, and memory formation; one or more molecules involved in regulating acute and chronic inflammations; and one or more molecules involved in tumor suppression or activation.

Clause 8. The method of any one of clauses 1-7, wherein the plurality of different molecules for the patient include one or more molecules identified using at least one of: one or more metabolomic biomarker panels, fMRI and/or PET-tracer enhanced imaging techniques, epigenetics, one or more blood biomarker panels, one or more of transcriptomics, proteomics, microbiomics, and genomics test (OMICs), and one or more early cancer biomarker panels.

Clause 9. The method of any one of clauses 1-8, wherein selecting the first intervention module and selecting a first intervention therein is performed based on an AI inference model.

Clause 10. The method of any one of clauses 1-9, comprising administering the selected first intervention as a treatment to the patient.

Clause 11. The method of clause 10, comprising, following administering the selected first intervention, updating the knowledge graph based on a feedback loop that applies data corresponding to one or more updated biomarker levels for the patient to adjust the knowledge graph by making an adjustment selected from: adding or removing a node, adding or removing a link, adjusting a link type, adjusting a weighting of a link.

Clause 12. The method of any one of clauses 1-11, wherein the knowledge graph comprises a plurality of links, each link connecting a set of nodes of a plurality of nodes in the knowledge graph, and wherein each of the plurality of links are weighted with one of a plurality of different link weights.

Clause 13. The method of any one of clauses 1-12, wherein the knowledge graph comprises a plurality of links, each link connecting a set of nodes of a plurality of nodes in the knowledge graph, and wherein the plurality of links comprises a plurality of different types of links that are traversed differently from one another in determining that at least one of the plurality of biomarker levels for the patient do not fall within the one or more target biomarker ranges and selecting the first intervention module using the knowledge graph.

Clause 14. A system comprising one or more processors and memory storing instructions configured to be executed by the one or more processors to cause the system to: obtain data representing a plurality of biomarker levels for a plurality of different molecules for a patient; determine, based on the obtained data representing the plurality of biomarker levels and using a knowledge graph linking one or more intervention modules, one or more target biomarker ranges, and one or more outcomes, whether at least one of the plurality of biomarker levels for the patient fall within the one or more target biomarker ranges; based on a determination that the at least one of the plurality of biomarker levels do not fall within the one or more target biomarker ranges, select, based on the obtained data and using the knowledge graph, a first one of the plurality of respective intervention modules associated with the at least one biomarker level that does not fall within the target biomarker range; select a first intervention associated with the selected first intervention module; and generate, by an outputter, an output indicating the selected first intervention.

Clause 15. The system of clause 14, wherein the instructions configured to be executed by the one or more processors cause the system to: select, based on the selected intervention module, one or more continuous monitoring tools from a set of continuous monitoring tools; and obtain updated biomarker data, via the one or more continuous monitoring tools, indicative of the changes in biomarker levels of the patient in response to the selected first intervention.

Clause 16. The system of clause 15, wherein the instructions configured to be executed by the one or more processors cause the system to: determine, after the changes in biomarker levels, whether the changed biomarker levels of the patient fall within the one or more target ranges; in response to a determination that the changed biomarker levels of the patient fall within the one or more target ranges, generate, by the outputter, a second output indicating a subsequent second intervention module and a selected second intervention therein.

Clause 17. The system of clause 16, wherein the instructions configured to be executed by the one or more processors cause the system to, in response to a determination that the changed biomarker levels of the patient do not fall within the one or more target ranges, generate, by the outputter, a third output indicating an escalated intervention within the first intervention module.

Clause 18. The system of any one of clauses 15-17, wherein the one or more continuous monitoring tools comprise one or more tools selected from: a survey and/or automated GUI-based data collection tool; a wearable device for monitoring blood glucose levels; a wearable device for monitoring cortisol levels; a wearable device for monitoring sleep quality, physical activity levels, stress levels, and/or emotional states correlating with high arousal states; and a wearable device for monitoring movement data, elevation data, and/or co-location or proximity data with other persons.

Clause 19. The system of any one of clauses 14-18, wherein the plurality of different molecules for the patient include one or more molecules selected from: Dopamine; Testosterone and/or Estrogen (which in some embodiments may be referred to and/or quantified as a testosterone level); Serotonin; Oxytocin; Arachidonoyl ethanolamine (AEA) and/or 2-archidonoyl glycerol (2-AG); beta-Endorphin; Cortisol; Adrenaline and/or Noradrenalin; Brain-derived neurotrophic factor (BDNF); and Eicosatetraenoic acid (EPA) and/or Docosahexaenoic acid (DHA).

Clause 20. The system of any one of clauses 14-19, wherein the plurality of different molecules for the patient include at least one of: one or more molecules involved in mitochondrial energy metabolism; one or more molecules involved in neuronal activation, enhanced cognition, and memory formation; one or more molecules involved in regulating acute and chronic inflammations; and one or more molecules involved in tumor suppression or activation.

Clause 21. The system of any one of clauses 14-20, wherein the plurality of different molecules for the patient include one or more molecules identified using at least one of: one or more metabolomic biomarker panels, fMRI and/or PET-tracer enhanced imaging techniques, epigenetics, one or more blood biomarker panels, one or more of transcriptomics, proteomics, microbiomics, and genomics test (OMICs), and one or more early cancer biomarker panels.

Clause 22. The system of any one of clauses 14-21, wherein selecting the first intervention module and selecting a first intervention therein is performed based on an AI inference model.

Clause 23. The system of any one of clauses 14-22, wherein the instructions configured to be executed by the one or more processors cause the system to update the knowledge graph based on a feedback loop that applies data corresponding to one or more updated biomarker levels for the patient to adjust the knowledge graph following the first invention by making an adjustment selected from: adding or removing a node, adding or removing a link, adjusting a link type, adjusting a weighting of a link.

Clause 24. The system of any one of clauses 14-23, wherein the knowledge graph comprises a plurality of links, each link connecting a set of nodes of a plurality of nodes in the knowledge graph, and wherein each of the plurality of links are weighted with one of a plurality of different link weights.

Clause 25. The system of any one of clauses 14-24, wherein the knowledge graph comprises a plurality of links, each link connecting a set of nodes of a plurality of nodes in the knowledge graph, and wherein the plurality of links comprises a plurality of different types of links that are traversed differently from one another in determining that at least one of the plurality of biomarker levels for the patient do not fall within the one or more target biomarker ranges and selecting the first intervention module using the knowledge graph.

Clause 26. A non-transitory computer-readable storage medium storing instructions which, when executed by a system comprising one or more processors, cause the system to: obtain data representing a plurality of biomarker levels for a plurality of different molecules for a patient; determine, based on the obtained data representing the plurality of biomarker levels and using a knowledge graph linking one or more intervention modules, one or more target biomarker ranges, and one or more outcomes, whether at least one of the biomarker levels for the patient do not fall within the one or more target biomarker ranges; based on a determination that the at least one of the plurality of biomarker levels do not fall within the one or more target biomarker ranges, select, based on the obtained data and using the knowledge graph, a first one of the plurality of respective intervention modules associated with the at least one biomarker level that does not fall within the target biomarker range; select a first intervention associated with the selected first intervention module; and generate, by an outputter, an output indicating the selected first intervention.

### Examples

The following examples are exemplary and are not intended to limit the scope of the disclosure provided herein.

### Prophetic Example—Sleep Module

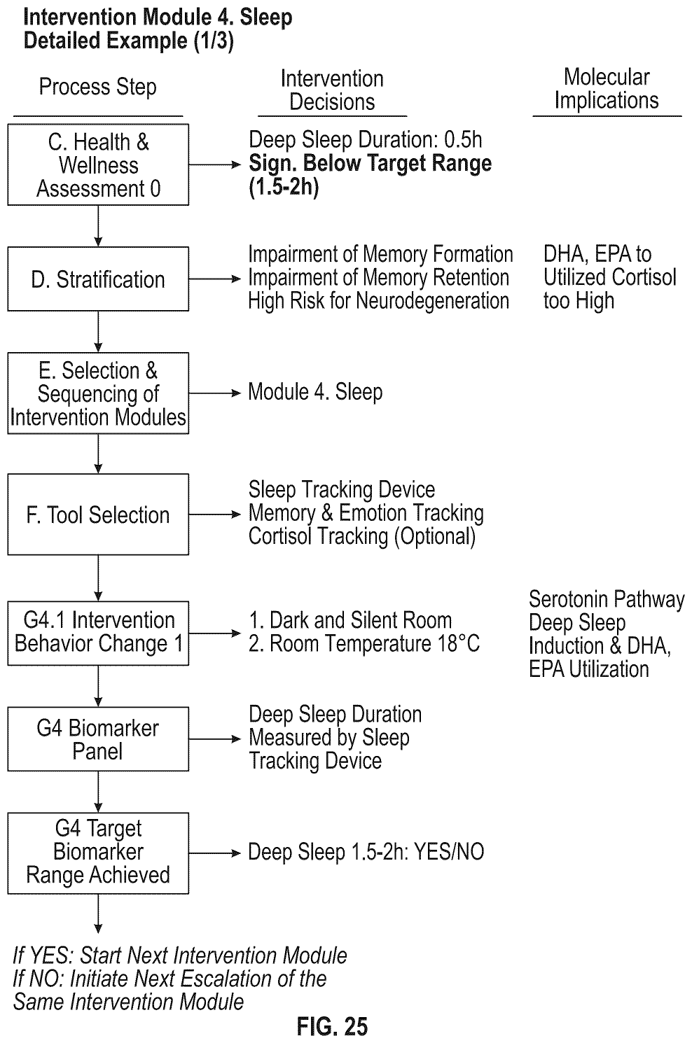

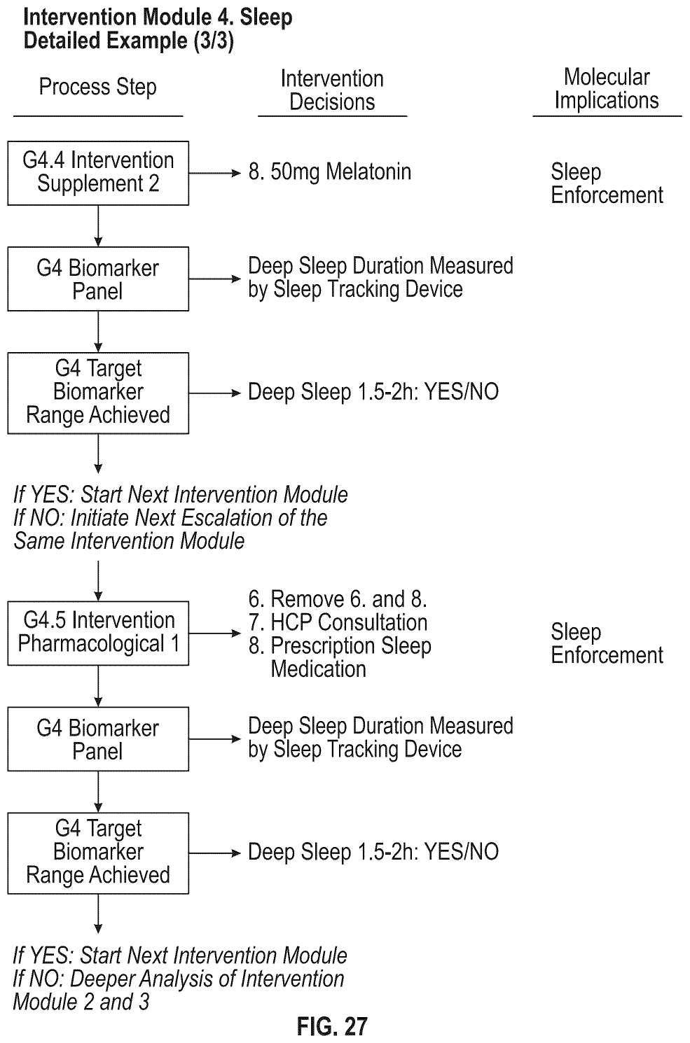

In a prophetic example shown in FIGS. 25-27, an initial health and wellness assessment is performed to gather biomarker data of the patient. The biomarker data is processed, and shows only 30 minutes of deep sleep instead of 90-120 minutes (the target range for this marker). Deep sleep is the period when memories are recorded and is directly linked to the availability of DHA, EPA, and cortisol (three of the ten molecules or molecule groups indicated above). The system stratifies the biomarker data to determine that the sleep biomarker data can impair memory formation and retention, and increases the risk for neurodegeneration.

Thus, the system selects the sleep intervention module based on the biomarker data processing. A sleep tracking device is selected as a continuous monitoring tool, and a tool (e.g., Matter app) for memory and emotion tracking is selected as well. Optionally, a cortisol patch can be selected for continuous monitoring.

An output is automatically generated to indicate to the patient which intervention module(s) and which interventions/treatments therein have been selected. The output may be electronically transmitted to a patient, used to update information stored in association with the patient, automatically rendered for display to a patient or caregiver, and/or used to automatically control an operation state of one or more automated devices (e.g., to automatically provide electronic coaching, automatically place a patient in communication with another human being (whether a family member, friend, or medical or mental health professional), and/or automatically administer a pharmacological treatment).

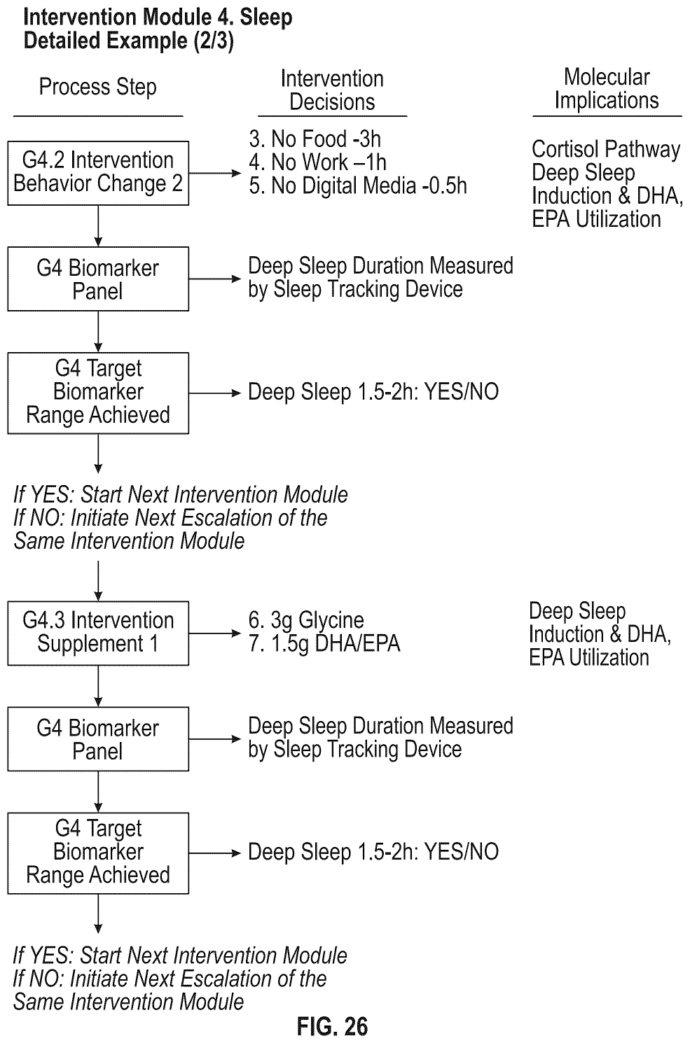

In this example, the interventions in the sleep module have a four-step intervention sequence. First, the patient is guided for a behavioral intervention of cooling the sleeping room to 18° C., complete darkness in the sleeping room and no digital media use 1 hour before going to bed. Continuous monitoring (e.g., a biomarker panel) is performed to assess whether the intervention is successful at achieving the target marker range. If, after two weeks, the deep sleep duration is in the target range, the sleep intervention module is completed, and the patient is progressed to the next module. If, after two weeks, the deep sleep duration is still not in the target range, the intervention is escalated by introducing an additional intervention on top of the first one, as shown in FIG. 26.

In this example, the next intervention in the sleep module is no food for 3 hours before sleep, no work for an hour before sleep, and no digital media for a half hour before sleep. Again, continuous monitoring is performed to assess whether the combination of interventions is successful at achieving the target range. If, after two weeks, the deep sleep duration is in the target range, the sleep intervention module is completed, and the patient is progressed to the next module. If, after two weeks, the deep sleep duration is still not in the target range, the intervention is escalated by introducing an additional intervention on top of the existing two.

In this example, the next intervention in the sleep module is a nutritional supplement intervention of 3g Glycine, plus 1.5g EPA/DHA oil to be taken 20 minutes before going to bed. Again, continuous monitoring is performed to assess whether the combination of interventions is successful at achieving the target range. If, after two weeks, the deep sleep duration is in the target range, the sleep intervention module is completed, and the patient is progressed to the next module. If, after two weeks, the deep sleep duration is still not in the target range, the intervention is escalated by introducing an additional intervention on top of the existing set of interventions.

In this example, the next intervention in the sleep module is an additional supplement intervention of 50 mg of Melatonin to be taken before bed. Again, continuous monitoring is performed to assess whether the combination of interventions is successful at achieving the target range. If, after two weeks, the deep sleep duration is in the target range, the sleep intervention module is completed, and the patient is progressed to the next module. If, after two weeks, the deep sleep duration is still not in the target range, the intervention is escalated by introducing an additional intervention on top of the existing set of interventions.

In this example, the next intervention in the sleep module is a pharmacologic intervention of prescription sleeping agent (e.g., Ambien) to be taken before bed, with healthcare professional consultation. Again, continuous monitoring is performed to assess whether the combination of interventions is successful at achieving the target range. If, after two weeks, the deep sleep duration is in the target range, the sleep intervention module is completed, and the patient is progressed to the next module. If, after two weeks, the deep sleep duration is still not in the target range, the system returns to interventions two and three for a deeper analysis.

### Working Example—AI-Based Report Generation, Including Intervention Determination

Using scaled biomarker data of a patient, as described herein, trained AI agents were used to score and generate an assessment of the patient's key biomolecules, and to determine interventions for maintaining and improving the biomolecules. The outputs of the AI agents are discussed below.

### Scoring Brief Module

The scoring brief module determined a good life (GL) score of 67.6 for the patient. The scoring brief module generated a narrative explaining the assigned score, including influence of the various molecules on the score. The scoring brief module then walked through individual scores for each of the molecules that contributed to the overall GL score-dopamine (37.8), BDNF (73.4), DHA (32.5), EPA (26.4), cortisol (73.8), adrenaline (78.8), estrogen (81), testosterone (75.7), serotonin (27.9), oxytocin (75), AEA (75.9), 2-AG (30.0), and beta endorphins (65.1)—with narratives explaining the assessed biomarkers that contributed to each of the scores.

### Canonical Pattern Module

The canonical pattern module identified physiological patterns based on the patient's biomarker data and generated narratives explaining how these identified patterns contribute to the patient's Good Life function/imbalance. For this patient, the physiological patterns identified by the AI agent included hyperglycemic-insulin dysregulation, high androgen tone/elevated SHBG, blunted dopaminergic salience with opioid bias, sleep fragmentation and REM deficit, contextual social bonding mismatch, low systemic inflammation with autoimmune signature, atherogenic lipoprotein burden, low NAD+ and magnesium strain, obstructive respiratory pattern with preserved aerobic capacity, and high drive/habitual screen use/burnout risk. For each of the identified physiological patterns, the AI agent generated a narrative explaining the pattern.

### Exploratory Pattern Module

The exploratory pattern module flagged physiological patterns that were not covered by the canonical pattern module and generated narratives for each that included biologically plausible hypotheses to explain them. The exploratory pattern module also determined whether each physiological pattern necessitated a deeper study. For this patient, the physiological patterns identified by the AI agent included iron-limited dopamine synthesis, tissue magnesium signaling deficit, hyperglycemia-driven NAD+depletion, peripheral hyperinsulinemia (leading to central insulin resistance), unexplained SHBG elevation, caregiving-engaged opioidergic tone, evening screen light suppresses REM, and low leptin signals and reward/arousal balance. For each of the physiological patterns, the AI agent generated a narrative including an observation, whether the pattern needed research, and a conclusion. Of the indicated physiological patterns, the AI agent determined that the unexplained SHBG elevation needed research, and in turn automatically generated a research prompt for doing so.

### Deep Research Module

The deep research module performed the research for the physiological patterns flagged by the exploratory pattern module. For this patient, that included an evidence review of the unexplained SHBG elevation. Using the prompt generated by the exploratory pattern module, the deep research module generated an evidence-backed narrative output on the SHBG elevation.

### Molecular Narrative Module

The molecular narrative module generated a final report including a narrative summarizing the patient's molecules in a format readable by the patient. The narrative included a breakdown of the scores assigned to each of the molecules that contributed to the overall GL score. For each of the molecules, the AI agent generated a unique narrative explaining the score assigned to the molecule. The molecular narrative module also generated a narrative explaining how the good life molecules can be supported, focusing on specific biomarkers that lend to the molecules.

### Recommendation Module

The recommendation module generated a personalized plan of next steps for the patient. For this patient, the module recommended urgent glycemic optimization with endocrine review, resolve nocturnal airway & a sleep study referral, phone curfew & a pickup reduction protocol, raise EPA/DHA via a diet and targeted supplement, intracellular magnesium repletion, iron optimization for dopamine synthesis, NAD+support via time-restricted eating and lifestyle, shift training timing to boost BDNF/dopamine, behavioral redesign to increase dopamine amplitude, and cardio-metabolic vascular review and lipid strategy. For each of these interventions, the AI agent generated a narrative explaining the intervention, necessary actions, and expected biomarker results for completing the intervention.

### CONCLUSION

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

For the purpose of clarity and a concise description, features are described herein as part of the same or separate examples; however, it will be appreciated that the scope of the disclosure includes examples having combinations of all or some of the features described.

What is claimed is:

1. A method to improve and maintain molecular biomarker levels in a patient, comprising:
obtaining biomarker data representing a plurality of biomarker levels for a plurality of different molecules for a patient;
determining, based on the obtained data representing the plurality of biomarker levels and using a knowledge graph linking one or more intervention modules, one or more target biomarker ranges, and one or more outcomes, that at least one of the plurality of biomarker levels for the patient do not fall within the one or more target biomarker ranges;
selecting, based on the obtained data and using the knowledge graph, a first one of the plurality of respective intervention modules associated with the at least one biomarker level that does not fall within the target biomarker range;
selecting a first intervention associated with the selected first intervention module; and
generating, by an outputter, an output indicating the selected first intervention.

2. The method of claim 1, comprising:
selecting, based on the selected intervention module, one or more continuous monitoring tools from a set of continuous monitoring tools; and
obtaining updated biomarker data, via the one or more continuous monitoring tools, indicative of the changes in biomarker levels of the patient in response to the selected first intervention.

3. The method of claim 2, comprising:
determining, after the changes in biomarker levels, that the changed biomarker levels of the patient fall within the one or more target ranges; and
in response to the determination that the changed biomarker levels of the patient fall within the one or more target ranges, generating, by the outputter, a second output indicating a subsequent second intervention module and a selected second intervention therein.

4. The method of claim 2, comprising:
determining, after the changes in biomarker levels, that the changed biomarker levels of the patient do not fall within the one or more target ranges; and
in response to the determination that the changed biomarker levels of the patient do not fall within the one or more target ranges, generating, by the outputter, a third output indicating an escalated intervention within the first intervention module.

5. The method of claim 2, wherein the one or more continuous monitoring tools comprise one or more tools selected from:
a survey and/or automated GUI-based data collection tool;
a wearable device for monitoring blood glucose levels;
a wearable device for monitoring cortisol levels;
a wearable device for monitoring sleep quality, physical activity levels, stress levels, and/or emotional states correlating with high arousal states; and
a wearable device for monitoring movement data, elevation data, and/or co-location or proximity data with other persons.

6. The method of claim 1, wherein the plurality of different molecules for the patient include one or more molecules selected from:
Dopamine;
Testosterone and/or Estrogen (which in some embodiments may be referred to and/or quantified as a testosterone level);
Serotonin;
Oxytocin;
Arachidonoyl ethanolamine (AEA) and/or 2-archidonoyl glycerol (2-AG);
beta-Endorphin;
Cortisol;
Adrenaline and/or Noradrenalin;
Brain-derived neurotrophic factor (BDNF); and
Eicosatetraenoic acid (EPA) and/or Docosahexaenoic acid (DHA).

7. The method of claim 1, wherein the plurality of different molecules for the patient include at least one of:
one or more molecules involved in mitochondrial energy metabolism;
one or more molecules involved in neuronal activation, enhanced cognition, and memory formation;
one or more molecules involved in regulating acute and chronic inflammations; and
one or more molecules involved in tumor suppression or activation.

8. The method of claim 1, wherein the plurality of different molecules for the patient include one or more molecules identified using at least one of:
one or more metabolomic biomarker panels,
fMRI and/or PET-tracer enhanced imaging techniques,
epigenetics,
one or more blood biomarker panels,
one or more of transcriptomics, proteomics, microbiomics, and genomics test (OMICs), and
one or more early cancer biomarker panels.

9. The method of claim 1, wherein selecting the first intervention module and selecting a first intervention therein is performed based on an AI inference model.

10. The method of claim 1, comprising administering the selected first intervention as a treatment to the patient.

11. The method of claim 10, comprising, following administering the selected first intervention, updating the knowledge graph based on a feedback loop that applies data corresponding to one or more updated biomarker levels for the patient to adjust the knowledge graph by making an adjustment selected from: adding or removing a node, adding or removing a link, adjusting a link type, adjusting a weighting of a link.

12. The method of claim 1, wherein the knowledge graph comprises a plurality of links, each link connecting a set of nodes of a plurality of nodes in the knowledge graph, and wherein each of the plurality of links are weighted with one of a plurality of different link weights.

13. The method of claim 1, wherein the knowledge graph comprises a plurality of links, each link connecting a set of nodes of a plurality of nodes in the knowledge graph, and wherein the plurality of links comprises a plurality of different types of links that are traversed differently from one another in determining that at least one of the plurality of biomarker levels for the patient do not fall within the one or more target biomarker ranges and selecting the first intervention module using the knowledge graph.

14. A system comprising one or more processors and memory storing instructions configured to be executed by the one or more processors to cause the system to:
obtain data representing a plurality of biomarker levels for a plurality of different molecules for a patient;
determine, based on the obtained data representing the plurality of biomarker levels and using a knowledge graph linking one or more intervention modules, one or more target biomarker ranges, and one or more outcomes, whether at least one of the plurality of biomarker levels for the patient fall within the one or more target biomarker ranges;
based on a determination that the at least one of the plurality of biomarker levels do not fall within the one or more target biomarker ranges, select, based on the obtained data and using the knowledge graph, a first one of the plurality of respective intervention modules associated with the at least one biomarker level that does not fall within the target biomarker range;
select a first intervention associated with the selected first intervention module; and
generate, by an outputter, an output indicating the selected first intervention.

15. The system of claim 14, wherein the instructions configured to be executed by the one or more processors cause the system to:
select, based on the selected intervention module, one or more continuous monitoring tools from a set of continuous monitoring tools; and
obtain updated biomarker data, via the one or more continuous monitoring tools, indicative of the changes in biomarker levels of the patient in response to the selected first intervention.

16. The system of claim 15, wherein the instructions configured to be executed by the one or more processors cause the system to:
determine, after the changes in biomarker levels, whether the changed biomarker levels of the patient fall within the one or more target ranges;
in response to a determination that the changed biomarker levels of the patient fall within the one or more target ranges, generate, by the outputter, a second output indicating a subsequent second intervention module and a selected second intervention therein.

17. The system of claim 16, wherein the instructions configured to be executed by the one or more processors cause the system to, in response to a determination that the changed biomarker levels of the patient do not fall within the one or more target ranges, generate, by the outputter, a third output indicating an escalated intervention within the first intervention module.

18. The system of claim 15, wherein the one or more continuous monitoring tools comprise one or more tools selected from:
a survey and/or automated GUI-based data collection tool;
a wearable device for monitoring blood glucose levels;
a wearable device for monitoring cortisol levels;
a wearable device for monitoring sleep quality, physical activity levels, stress levels, and/or emotional states correlating with high arousal states; and
a wearable device for monitoring movement data, elevation data, and/or co-location or proximity data with other persons.

19. The system of claim 14, wherein the plurality of different molecules for the patient include one or more molecules selected from:
Dopamine;
Testosterone and/or Estrogen (which in some embodiments may be referred to and/or quantified as a testosterone level);
Serotonin;
Oxytocin;
Arachidonoyl ethanolamine (AEA) and/or 2-archidonoyl glycerol (2-AG);
beta-Endorphin;
Cortisol;
Adrenaline and/or Noradrenalin;
Brain-derived neurotrophic factor (BDNF); and
Eicosatetraenoic acid (EPA) and/or Docosahexaenoic acid (DHA).

20. The system of claim 14, wherein the plurality of different molecules for the patient include at least one of:
one or more molecules involved in mitochondrial energy metabolism;
one or more molecules involved in neuronal activation, enhanced cognition, and memory formation;
one or more molecules involved in regulating acute and chronic inflammations; and
one or more molecules involved in tumor suppression or activation.

21. The system of claim 14, wherein the plurality of different molecules for the patient include one or more molecules identified using at least one of:
one or more metabolomic biomarker panels,
fMRI and/or PET-tracer enhanced imaging techniques,
epigenetics,
one or more blood biomarker panels,
one or more of transcriptomics, proteomics, microbiomics, and genomics test (OMICs), and
one or more early cancer biomarker panels.

22. The system of claim 14, wherein selecting the first intervention module and selecting a first intervention therein is performed based on an AI inference model.

23. The system of claim 14, wherein the instructions configured to be executed by the one or more processors cause the system to update the knowledge graph based on a feedback loop that applies data corresponding to one or more updated biomarker levels for the patient to adjust the knowledge graph following the first invention by making an adjustment selected from: adding or removing a node, adding or removing a link, adjusting a link type, adjusting a weighting of a link.

24. The system of claim 14, wherein the knowledge graph comprises a plurality of links, each link connecting a set of nodes of a plurality of nodes in the knowledge graph, and wherein each of the plurality of links are weighted with one of a plurality of different link weights.

25. The system of claim 14, wherein the knowledge graph comprises a plurality of links, each link connecting a set of nodes of a plurality of nodes in the knowledge graph, and wherein the plurality of links comprises a plurality of different types of links that are traversed differently from one another in determining that at least one of the plurality of biomarker levels for the patient do not fall within the one or more target biomarker ranges and selecting the first intervention module using the knowledge graph.

26. A non-transitory computer-readable storage medium storing instructions which, when executed by a system comprising one or more processors, cause the system to:
obtain data representing a plurality of biomarker levels for a plurality of different molecules for a patient;
determine, based on the obtained data representing the plurality of biomarker levels and using a knowledge graph linking one or more intervention modules, one or more target biomarker ranges, and one or more outcomes, whether at least one of the biomarker levels for the patient do not fall within the one or more target biomarker ranges;
based on a determination that the at least one of the plurality of biomarker levels do not fall within the one or more target biomarker ranges, select, based on the obtained data and using the knowledge graph, a first one of the plurality of respective intervention modules associated with the at least one biomarker level that does not fall within the target biomarker range;
select a first intervention associated with the selected first intervention module; and
generate, by an outputter, an output indicating the selected first intervention.
